(19) United States
(12) Patent Application Publication
(10) Pub. No.: US 2018/0028264 A1
(43) Pub. Date: Feb. 1, 2018

(54) RADIO-FREQUENCY ELECTRICAL MEMBRANE BREAKDOWN FOR THE TREATMENT OF CARDIAC RHYTHM DISORDERS AND FOR RENAL NEUROMODULATION

(71) Applicant: RFEMB Holdings, LLC, North Brandford, CT (US)

(72) Inventors: Gary M. Onik, Ft. Lauderdale, FL (US); James A. Miessau, Branford, CT (US); David G. Bostwick, Orlando, FL (US)

(21) Appl. No.: 15/549,194

(22) PCT Filed: Feb. 8, 2016
(86) PCT No.: PCT/US16/16955

§ 371 Date: Aug. 7, 2017

Related U.S. Application Data
(60) Provisional application No. 62/112,742, filed on Feb. 6, 2015.
(60) Provisional application No. 62/112,844, filed on Feb. 6, 2015.

(51) Int. Cl. A61B 18/14

(52) U.S. Cl. CPC A61B 18/1492; A61B 2018/0022

(57) ABSTRACT

An imaging, guidance, planning and treatment system integrated into a single unit or assembly of components, and a method for using same, that can be safely and effectively deployed to treat cardiac rhythm disorders and atrial fibrillation in an open operative procedure, or in a minimally invasive thorascopic surgical procedure, or in a transvascular procedure. The system utilizes the novel process of Radio-Frequency Electrical Membrane Breakdown (“EMB” or “RFEMB”) to destroy the cellular membranes of targeted cardiac tissue to create transmural lesions designed to prevent atrial reentry and to allow sinus impulses to activate the atrial myocardium thereby preserving atrial transport and aiding its function. The system preferably comprises at least one EMB treatment probe 20 and at least one controller unit for at least partially automating the treatment process.

### CROSS-REFERENCE TO RELATED APPLICATIONS

The present invention is a continuation of U.S. Provisional Patent Application Ser. Nos. 62/112,742, filed Feb. 6, 2015, and 62/112,844, filed Feb. 6, 2015, both of which are continuations-in-part of U.S. patent application Ser. No. 14/451,333, filed Aug. 4, 2014, which claims priority to U.S. Provisional Patent Application Nos. 61/912,172, filed Dec. 5, 2013, 61/861,565, filed Aug. 2, 2013, and 61/867,048, filed Aug. 17, 2013, all of which are incorporated herein by reference.

### BACKGROUND OF THE INVENTION

### 1. Field of the Invention

The present invention relates generally to medical devices and treatment methods, and more particularly, to a device and methods of utilizing radio frequency electrical membrane breakdown (“RFEMB”, or “EMB”) for reducing sympathetic renal nerve activity and treating atrial fibrillation and other cardiac arrhythmias.

### 2. Background of the Invention

Atrial arrhythmia, or irregular heartbeat, corresponds to three separate detrimental sequela: (1) a change in the ventricular response, including the onset of an irregular ventricular rhythm and an increase in ventricular rate; (2) detrimental hemodynamic consequences resulting from loss of atrioventricular synchrony, decreased ventricular filling time, and possible atrioventricular valve regurgitation; and (3) an increased likelihood of sustaining a thromboembolic event because of loss of effective contraction and atrial stasis of blood in the left atrium. Atrial arrhythmia may be treated using several methods. Pharmacological treatment of atrial fibrillation, for example, is initially the preferred approach, first to maintain normal sinus rhythm, or secondly to decrease the ventricular response rate. While these medications may reduce the risk of thrombus collecting in the atrial appendages if the atrial fibrillation can be converted to sinus rhythm, this form of treatment is not always effective. Patients with continued atrial fibrillation and only ventricular rate control continue to suffer from irregular heartbeats and from the effects of altered hemodynamics due to the lack of normal sequential atrioventricular contractions, as well as continue to face a significant risk of thromboembolism.

Other forms of treatment include chemical cardioversion to normal sinus rhythm, electrical cardioversion, and radio frequency (RF) catheter ablation of selected areas determined by mapping. In the more recent past, other surgical procedures have been developed for atrial fibrillation, including left atrial isolation, transvenous catheter or cryosurgical ablation of His bundle, and the Corridor procedure, which have effectively eliminated irregular ventricular rhythm. However, these procedures have for the most part failed to restore normal cardiac hemodynamics, or alleviate the patient's vulnerability to thromboembolism because the atria are allowed to continue to fibrillate. Accordingly, a more effective surgical treatment is required to cure medically refractory atrial fibrillation of the heart.

On the basis of electrophysiologic mapping of the atria and identification of reentrant circuits, a surgical approach was developed which effectively creates an electrical maze in the atrium (i.e., the MAZE procedure) and precludes the ability of the atria to fibrillate.

Briefly, in the procedure commonly referred to as the MAZE III procedure, strategic atrial incisions are performed to prevent atrial reentry and allow sinus impulses to activate the atrial myocardium, thereby preserving atrial transport function postoperatively. Since atrial fibrillation is characterized by the presence of multiple macroreentrant circuits that are fleeting in nature and can occur anywhere in the atria, it is prudent to interrupt all of the potential pathways for atrial macroreentrant circuits. These circuits, incidentally, have been identified by intraoperative mapping both experimentally and clinically in patients.

Generally, this procedure includes the excision of both atrial appendages, and the electrical isolation of the pulmonary veins. Further, strategically placed atrial incisions not only interrupt the conduction routes of the most common reentrant circuits, but they also direct the sinus impulse from the sinoatrial node to the atrioventricular node along a specified route. In essence, the entire atrial myocardium, with the exception of the atrial appendages and the pulmonary veins, is electrically activated by providing for multiple blind alleys off the main conduction route between the sinoatrial node to the atrioventricular node. Atrial transport function is thus preserved postoperatively, as generally set forth in the series of articles. See Cox, Schuessler, Boineau, Canavan, Cain, Lindsay, Stone, Smith, Corr, Chang, and D'Agostino, Jr., The Surgical Treatment of Atrial Fibrillation (pts. 1-4), 101 THORAC CARDIOVASC SURG., 402-426, 569-592 (1991).

While the MAZE III procedure has proven effective in ablating medically refractory atrial fibrillation and associated detrimental sequela, this operational procedure is traumatic to the patient since substantial incisions are introduced into the interior chambers of the heart. Moreover, using current techniques, many of these procedures require a gross thoracotomy, usually in the form of a median sternotomy, to gain access into the patient's thoracic cavity. A saw or other cutting instrument is used to cut the sternum longitudinally, allowing two opposing halves of the anterior or ventral portion of the rib cage to be spread apart.

A large opening into the thoracic cavity is thus created, through which the surgical team may directly visualize and operate upon the heart for the MAZE III procedure. Such a large opening further enables manipulation of surgical instruments and/or removal of excised heart tissue since the surgeon can position his or her hands within the thoracic cavity in close proximity to the exterior of the heart. The patient is then placed on cardiopulmonary bypass to maintain peripheral circulation of oxygenated blood.

Not only is the MAZE III procedure itself traumatic to the patient, but the postoperative pain and extensive recovery time due to the conventional thoracotomy substantially increase trauma and further extend hospital stays. Moreover, such invasive, open-chest procedures significantly increase the risk of complications and the pain associated with sternal incisions. Therefore, the Maze III procedure is often reserved for patients with atrial fibrillation that are already having an open heart operation.

Improvements in the Maze III procedure have been made in an effort to replace the surgical incisions required into the cardiac muscle, which has lead to a recent resurgence of the field of surgical ablation for the treatment of atrial fibrillation, predominantly based on a renewed interest in energy sources that create lesions via thermal injury.

The majority of currently used energy sources utilize hyperthermic injury by obtaining a tissue temperature of 50° C., which has been shown to be the temperature at which electrophysiologic disruption occurs. A variety of energy sources are used to induce hyperthermic damage including radiofrequency (RF), microwave, laser, and high-intensity focal ultrasound devices. Se Viola N, Williams M R, Oz M C, Ad N. 2002, “The technology in use for the surgical ablation of atrial fibrillation”, Semin Thorac Cardiovasc Surg 14:198-205.; Cummings J E, Pacifico A, Drago J L, Kilicaslan F, Natale A. 2005, “Alternative energy sources for the ablation of arrhythmias”, Pacing Clin Elec-trophysiol 28:434-43.; Ninet J, Roques X, Seitelberger R, et al. 2005, “Surgical ablation of atrial fibrillation with off-pump, epicardial, high-intensity focused ultrasound: results of a multicenter trial”, J Thorac Cardiovasc Surg 130:803-9).

Alliteratively, hypothermic injury of the atrial tissue has long been used with cryoablation devices, achieving injury at a tissue temperature of −55° C. While all of these energy sources have been widely utilized with varying results, (Barnett S D, Ad N. 2006, “Surgical ablation as treatment for the elimination of atrial fibrillation: a meta-analysis”, J Thorac Cardiovasc Surg 131:1029-35.) they do not always produce the required transmural lesion.

Furthermore, their use is time consuming in procedures in which time is of the essence. In addition, local complications due to overheating, tissue coagulation, and the variable temperature distribution in the treated tissue, which is typical to the fundamental physical characteristics of the heat-transfer process, have been reported. See Doll N, Borger M A, Fabricius A, et al. 2003, “Esophageal perforation during left atrial radiofrequency ablation: is the risk too high?” J Thorac Cardiovasc Surg 125:836-42.

Although atrial fibrillation may occur alone, this arrhythmia often associates with numerous cardiovascular conditions, including congestive heart failure (CHF), hypertensive cardiovascular disease, myocardial infarction, rheumatic heart disease and stroke. CHF is a condition that occurs when the heart becomes damaged and reduces blood flow to the organs of the body. If blood flow decreases sufficiently, kidney function becomes altered, which results in fluid retention, abnormal hormone secretions and increased constriction of blood vessels. These results increase the workload of the heart and further decrease the capacity of the heart to pump blood through the kidneys and circulatory system.

It is believed that progressively decreasing perfusion of the kidneys is a principal non-cardiac cause perpetuating the downward spiral of CHF. Moreover, the fluid overload and associated clinical symptoms resulting from these physiologic changes result in additional hospital admissions, poor quality of life and additional costs to the health care system.

In addition to their role in the progression of CHF, the kidneys play a significant role in the progression of Chronic Renal Failure (“CRF”), End-Stage Renal Disease (“ESRD”), hypertension (pathologically high blood pressure) and other cardio-renal diseases. The functions of the kidneys can be summarized under three broad categories: filtering blood and excreting waste products generated by the body's metabolism; regulating salt, water, electrolyte and acid-base balance; and secreting hormones to maintain vital organ blood flow.

Without properly functioning kidneys, a patient will suffer water retention, reduced urine flow and an accumulation of waste toxins in the blood and body. These conditions result from reduced renal function or renal failure (kidney failure) and are believed to increase the workload of the heart. In a CHF patient, renal failure will cause the heart to further deteriorate as fluids are retained and blood toxins accumulate due to the poorly functioning kidneys.

It has been established in animal models that the heart failure condition results in abnormally high sympathetic activation of the kidneys. An increase in renal sympathetic nerve activity leads to decreased removal of water and sodium from the body, as well as increased renin secretion. Increased renin secretion leads to vasoconstriction of blood vessels supplying the kidneys which causes decreased renal blood flow. Reduction of sympathetic renal nerve activity, e.g., via denervation, may reverse these processes.

Methods and apparatus for achieving renal neuromodulation, e.g., via localized drug delivery (such as by a drug pump or infusion catheter) or via use of a stimulation electric field, have been described as well in U.S. patent application Ser. No. 10/408,665, filed Apr. 8, 2003, and U.S. Pat. No. 6,978,174. In addition, methods and apparatus for treating renal disorders by applying a pulsed electric field to neural fibers that contribute to renal function and affecting the renal nerve activity by the mechanism of irreversible electroporation have been described in, for example, U.S. patent application Ser. No. 11/129,765, filed on May 13, 2005, and Ser. No. 11/189,563, filed on Jul. 25, 2005.

A pulsed electric field (“PEF”) may initiate renal neuromodulation, e.g., denervation, for example, via irreversible electroporation or via electrofusion. The PEF may be delivered from an apparatus positioned intravascularly, extravascularly, intra-to-extravascularly or a combination thereof.

Electrofusion comprises fusion of neighboring cells induced by exposure to an electric field. Contact between target neighboring cells for the purposes of electrofusion may be achieved in a variety of ways, including, for example, via dielectrophoresis. In tissue, the target cells may already be in contact, thus facilitating electrofusion.

Electroporation and electropermeabilization are methods of manipulating the cell membrane or intracellular apparatus. For example, the porosity of a cell membrane may be increased by inducing a sufficient voltage across the cell membrane through, e.g., short, high-voltage pulses. The extent of porosity in the cell membrane (e.g., size and number of pores) and the duration of effect (e.g., temporary or permanent) are a function of multiple variables, such as field strength, pulse width, duty cycle, electric field orientation, cell type or size and/or other parameters.

Cell membrane pores will generally close spontaneously upon termination of relatively lower strength electric fields or relatively shorter pulse widths (herein defined as “reversible electroporation”). However, each cell or cell type has a critical threshold above which pores do not close such that pore formation is no longer reversible; this result is defined as “irreversible electroporation,” (IRE) “irreversible breakdown” or “irreversible damage.”

IRE is a modality in which microsecond electrical pulses are applied across the cell to generate a destabilizing electric potential across biological membranes and cause the formation of nanoscale pores in the lipid bilayer; these defects are permanent and lead to cell death. In preliminary research, it has been shown that IRE is an independent modality from thermal modalities and that it affects tissue in a way that is different from conventional thermal ablation modalities. IRE leads to tissue death through an unusual path by producing nanoscale pores in the cell membrane only and sparing other tissue components, including macromolecules, proteins, connective tissue, and cell and tissue scaffold. The cell death is caused by the departure from homeostatic conditions inside the cell. The parameters of IRE are precise; i.e., an electrical pulse either causes IRE on the cell membrane or not, thereby producing sharp, cell-scale borders between affected and unaffected regions of tissues. It is not affected by blood flow and is capable of producing permanent non-thermal damage to tissue within a fraction of a second.

Irreversible electroporation relies on the phenomenon of electroporation. With reference to FIG. 1, electroporation refers to the fact that the plasma membrane of a cell exposed to high voltage pulsed electric fields within certain parameters, becomes temporarily permeable due to destabilization of the lipid bilayer and the formation of pores P. The cell plasma membrane consists of a lipid bilayer with a thickness t of approximately 5 nm. With reference to FIG. 2(A), the membrane acts as a nonconducting, dielectric barrier forming, in essence, a capacitor. Physiological conditions produce a natural electric potential difference due to charge separation across the membrane between the inside and outside of the cell even in the absence of an applied electric field. This resting transmembrane potential V′m ranges from 40 mv for adipose cells to 85 mv for skeletal muscle cells and 90 mv cardiac muscle cells and can vary by cell size and ion concentration among other things.

With continued reference to FIGS. 2(B)-2(D), exposure of a cell to an externally applied electric field E induces an additional voltage V across the membrane as long as the external field is present. The induced transmembrane voltage is proportional to the strength of the external electric field and the radius of the cell. Formation of transmembrane pores P in the membrane occurs if the cumulative resting and applied transmembrane potential exceeds the threshold voltage which may typically be between 200 mV and 1 V. Poration of the membrane is reversible if the transmembrane potential does not exceed the critical value such that the pore area is small in relation to the total membrane surface. In such reversible electroporation, the cell membrane recovers after the applied field is removed and the cell remains viable. Above a critical transmembrane potential and with longer exposure times, poration becomes irreversible leading to eventual cell death due an influx of extracellular ions resulting in loss of homeostasis and subsequent apoptosis. Pathology after irreversible electroporation of a cell does not show structural or cellular changes until 24 hours after field exposure except in certain very limited tissue types. However, in all cases the mechanism of cellular destruction and death by IRE is apoptotic which requires considerable time to pass and is not visible pathologically in a time frame to be clinically useful in determining the efficacy of IRE treatment which is an important clinical drawback to the method.

Irreversible electroporation (IRE) as an ablation method grew out of the realization that the “failure” to achieve reversible electroporation could be utilized to selectively kill undesired tissue. IRE effectively kills a predictable treatment area without the drawbacks of thermal ablation methods that destroy adjacent vascular and collagen structures. During a typical IRE treatment, one to three pairs of electrodes are placed in or around the tissue. Electrical pulses carefully chosen to induce an electrical field strength above the critical transmembrane potential are delivered in groups of 10, usually for nine cycles. Each 10-pulse cycle takes about one second, and the electrodes pause briefly before starting the next cycle. As described in U.S. Pat. No. 8,048,067 to Rubinsky, et. al and U.S. patent application Ser. No. 13/332,133 by Arena, et al. which are incorporated here by reference, the field strength and pulse characteristics are chosen to provide the necessary field strength for IRE but without inducing thermal effects as with RF thermal ablation.

However, the DC pulses used in currently available IRE methods and devices have characteristics that can limit their use or add risks for the patient because current methods and devices create severe muscle contraction during treatment. This is a significant disadvantage because it requires that a patient be placed and supported under general anesthesia with neuromuscular blockade in order for the procedure to be carried out, and this carries with it additional substantial inherent patient risks and costs. Moreover, since even relatively small muscular contractions can disrupt the proper placement of IRE electrodes, the efficacy of each additional pulse train used in a therapy regimen may be compromised without even being noticed during the treatment session. An addition limitation of IRE is that the DC pulses needed to create the IRE lesion cause electrical arcing, resulting in sparking at the juncture of the insulation and the active portion of the electrode, as well as between the electrodes when placed close together. Such arcing and its associated barotrauma have been shown to cause tissue perforation. Thus, it was felt that IRE might be inherently unsafe for such use in the clinical setting. Moreover, the lack of immediacy of results and the tendency for the tissue impedance to rise again as pores in the membrane close over time (which can clinically take 10 minutes and can continue for much longer) makes monitoring of tissue impedance not reliable for determination of efficacy of IRE treatment in this setting. The clinical use in patients of IRE for the treatment of atrial arrhythmia or reduction of sympathetic renal nerve activity has never been reported in the literature.

What is needed is a method for treating atrial fibrillation and other cardiac arrhythmias by creating transmural lesions in cardiac tissue to interrupt targeted electrophysiolgical pathways to control atrial fibrillation, and that avoids the risks of thermal trauma to cardiac tissue.

What is also needed is a method for achieving renal neuromodulation by creating lesions in renal nerves and neural fiber tissue to reduce sympathetic nerve activity.

In addition, an ablation method that can be accurately targeted at specific areas of cardiac and/or renal nerve tissue, and that preserves the cardiac structure or adjacent vascular tissue in the focal treatment area, would be advantageous.

It would also be advantages to provide a system that can be used in an open operative setting, in which the cardiac or renal nerve tissue can be ablated using RFEMB so as to create the desired transmural or renal nerve lesions.

It would also be advantageous to provide a system using an ablation modality with the ability to create and monitor cardiac tissue destruction using a thorascopic approach through methods that do not have the inherent limitations of IRE, does not require neuromuscular blockade, and does not cause potentially dangerous sparking, which would provide a minimally invasive surgical means for treating atrial fibrillation.

It would also be advantageous to provide a system using an ablation modality with the ability to create and monitor renal nerve tissue destruction using a laparascopic approach through methods that do not have the inherent limitations of IRE, does not require neuromuscular blockade, and does not cause potentially dangerous sparking, which would provide a minimally invasive surgical means for achieving renal neuromodulation.

It would also be advantageous to provide a system and method for carrying out this treatment under local anesthesia, using a method that does not require general anesthesia or a neuromuscular blockade.

### SUMMARY OF THE INVENTION

It is, therefore, an object of the present invention to provide a method that could be used for creating transmural lesions in cardiac tissue to control atrial fibrillation and other atrial and ventricular arrhythmias that avoids the risks of thermal trauma to cardiac tissue via tissue ablation using electrical pulses which cause immediate cell death through the mechanism of complete break down of the cellular membrane of the targeted tissue cells.

It is also an object of the present invention to provide a method that could be used for creating lesions in renal nerve tissue to create renal neuromodulation that avoids the risks of thermal trauma to adjacent vascular tissue via tissue ablation using electrical pulses which cause immediate cell death through the mechanism of complete breakdown of the cellular membrane of the targeted tissue cells.

It is another object of the present invention to provide such a treatment method that does not require the administration of general anesthesia or a neuromuscular blockade to the patient, so as to provide a system and method for carrying out this treatment in a minimally invasive procedure.

It is another object of the present invention to provide a treatment for atrial fibrillation and other atrial and ventricular arrhythmias with treatment probes through a transvascular route using a flexible catheter under imaging guidance.

It is another object of the present invention to provide a treatment for providing renal neuromodulation with treatment probes through a percutaneous approach using a flexible catheter under imaging guidance.

It is another object of the present invention to provide a system and method for creating neuromodulation to treat congestive heart failure, hypertension and other disorders with heightened sympathetic tone.

It is another object of the present invention to provide such a treatment method that can be used in an open operating setting, with full surgical access to the cardiac region, renal artery or renal nerve.

It is another object of the present invention to configure the delivery electrodes in such a way as to facilitate the use of the system in a minimally invasive operation carried out by thoracoscopy.

It is another object of the present invention to configure the delivery electrodes in such a way as to facilitate the use of the system in a minimally invasive operation carried out using a laproscopic approach.

The present invention is an imaging, guidance, planning and treatment system integrated into a single unit or assembly of components, and a method for using same, that can be safely and effectively deployed to treat atrial fibrillation and achieve renal neuromodulation with EMB treatment probes applied to the heart or in proximity to sympathetic renal nerve tissue. The invention is comprised of a combination of software, hardware and a process for employing the same through an endoscopic, endoscopic ultrasound, or imaging guided (CT, US, MRI, Flouroscopy) transvascular approach. The system utilizes the novel process of Radio-Frequency Electrical Membrane Breakdown (“EMB” or “RFEMB”) to ablate the cellular membranes of targeted cardiac or renal nerve tissue.

The use of EMB to achieve focal tumor ablation is disclosed in U.S. patent application Ser. No. 14/451,333 and International Patent Application No. PCT/US14/68774, which are both fully incorporated herein by reference.

EMB is the application of an external oscillating electric field to cause vibration and flexing of the cell membrane, which results in a dramatic and immediate mechanical tearing, disintegration and/or rupturing of the cell membrane. Unlike the IRE process, in which nanopores are created in the cell membrane but through which little or no content of the cell is released, EMB completely tears open the cell membrane such that the entire contents of the cell are expelled into the extracellular fluid, and internal components of the cell membrane itself are exposed. EMB achieves this effect by applying specifically configured electric field profiles, comprising significantly higher energy levels (as much as 100 times greater) as compared to the IRE process, to directly and completely disintegrate the cell membrane rather than to electroporate the cell membrane. Such electric field profiles are not possible using currently available IRE equipment and protocols. The inability of current IRE methods and energy protocols to deliver the energy necessary to cause EMB explains why IRE treated specimens have never shown the pathologic characteristics of EMB treated specimens, and is a critical reason why EMB had not until now been recognized as an alternative method of cell destruction.

The system according to the present invention comprises a software and hardware system, and method for using the same, for delivering EMB treatment to a target area, so that lesions of the size and shape needed result as the cells in the area are ablated. The system provides proprietary predictive software tools for designing an EMB treatment protocol to ablate said targeted tissue, and for applying said EMB treatment protocol to create the planned ablation. The system includes an EMB pulse generator 16, one or more EMB treatment probes 20, and one or more temperature probes 22. The system further employs a software-hardware controller unit (SHCU) operatively connected to said generator 16, probes 20, and temperature probe(s) 22, along with one or more optional devices such as endoscopic or US imaging scanners, ultrasound scanners, and/or other imaging devices or energy sources, and operating software for controlling the operation of each of these hardware devices.

In addition, a method of creating transmural cardiac lesions that can achieve electrical isolation of atrial tissue in an open operative setting such as the MAZE III procedure is disclosed.

In addition, a method of creating renal nerve lesions that can achieve neuromodulation in the sympathetic nerve adjacent to the renal arteries in an open operative setting is disclosed.

EMB, by virtue of its bipolar wave forms in the described frequency range, does not cause muscle twitching and contraction. Therefore a procedure using the same may be carried out under local anesthesia without the need for general anesthesia and neuromuscular blockade to attempt to induce paralysis during the procedure. Rather, anesthesia can be applied locally for the control of pain without the need for the deeper and riskier levels of sedation.

In addition, the energy profiles that are used to create EMB also avoid potentially serious patient risks from interference with cardiac sinus rhythm.

In addition, EMB, with the applied electrical parameters, does not cause sparking therefore eliminating the possibility of barotrauma that are associated with IRE.

### BRIEF DESCRIPTION OF THE DRAWINGS

FIG. 1 is a diagram of a cell membrane pore.

FIG. 2 is a diagram of cell membrane pore formation by a prior art method.

FIG. 3 is a schematic diagram of the software and hardware system according to the present invention.

FIG. 4A is a comparison of a prior art charge reversal with an instant charge reversal according to the present invention.

FIG. 4B is a square wave from instant charge reversal pulse according to the present invention.

FIG. 5 is a diagram of the forces imposed on a cell membrane as a function of electric field pulse width according to the present invention.

FIG. 6 is a diagram of a prior art failure to deliver prescribed pulses due to excess current.

FIG. 7A is a composite (1 and 2) of a schematic diagram depicting a US scan of a targeted tissue area.

FIG. 7B is a composite (1 and 2) of a schematic diagram depicting the results of a 3D Fused Image of the intended treatment area.

FIG. 8 is a composite (1 and 2) of a schematic diagram depicting the target treatment area and Predicted Ablation Zone relative to a therapeutic EMB treatment probe 20 prior to delivering treatment.

FIG. 9 is a schematic diagram of a pulse generation and delivery system for application of the method of the present invention.

FIG. 10 is a diagram of the parameters of a partial pulse train according to the present invention.

FIG. 11 is a composite (1, corresponding to cardiac treatment, and 2 corresponding to renal nerve treatment) of a schematic diagram depicting the target treatment area and Predicted Ablation Zone relative to a therapeutic EMB treatment probe 20 at the start of treatment delivery.

FIG. 12 is a composite (1, corresponding to cardiac treatment, and 2 corresponding to renal nerve treatment) of a schematic diagram depicting the positioning of a therapeutic EMB treatment probe 20 comprising an electromagnetic sensor/transmitter 26 according to an embodiment of the present invention proximate the treatment area 2 inside the cardiac chamber (FIG. 12(1)) and a blood vessel 401 (FIG. 12(2)).

FIG. 13 is a composite (1, corresponding to cardiac treatment, and 2 corresponding to renal nerve treatment) of a schematic diagram depicting the positioning of a therapeutic EMB treatment probe 20 comprising a thermocouple 7 according to another embodiment of the present invention proximate the treatment area 2 inside the cardiac chamber FIG. 12(1)) and a blood vessel 401 (FIG. 12(2)).

FIG. 14 is a composite (1 and 2) of a schematic diagram depicting the positioning of a therapeutic EMB treatment probe 20 comprising a thermocouple 7 according to another embodiment of the present invention.

FIG. 15 is a composite (1 and 2) of a schematic diagram depicting the positioning of a therapeutic EMB treatment probe 20 comprising a unipolar electrode 11 according to another embodiment of the present invention proximate the treatment area 2.

FIG. 16 is a schematic diagram depicting the positioning of a therapeutic EMB treatment probe 20 comprising an expandable electrode-bearing balloon 27 according to another embodiment of the present invention in the orifice of a pulmonary vein.

FIG. 17 is a schematic diagram depicting the positioning of a therapeutic EMB treatment probe 20 comprising an electrode-bearing expandable balloon 27 according to another embodiment of the present invention inside in the orifice of a pulmonary vein.

FIG. 18 is a schematic diagram depicting the positioning of a therapeutic EMB treatment probe 20 comprising an insulating sheath 23 bearing electrode 4 according to another embodiment of the present invention in a cardiac chamber.

FIG. 19 is a schematic diagram of clamp-type electrodes 20 according to another embodiment of the present invention.

FIG. 20 is a schematic diagram of the clamp-type electrodes 20 as shown in FIG. 19 further comprising an insulating member 43 to shield certain areas of the patient's body from electrical contact.

FIG. 21 is a schematic diagram of the clamp-type electrodes 20 with insulating member 43 as shown in FIG. 20 including perpendicular projection 43a.

FIG. 22 is a schematic diagram of the clamp-type electrodes 20 as shown in FIG. 19 with a multiplicity of small electrode members 3 interspersed with sensing electrodes 3a.

FIG. 23 is a schematic diagram of the clamp-type electrodes 20 as shown in FIG. 22 where insulating member 43 replace sensing electrodes 3a.

FIG. 24 is a composite (1 and 2) of an illustration of various tissue sizes with corresponding voltage strengths for treatment.

FIG. 25 is a schematic diagram of the clamp-type electrodes 20 as shown in FIG. 22 further comprising cannula 44 to ease insertion of probe 20 into a patient.

FIG. 26 is a schematic diagram of handheld a probe 20 according to another embodiment of the present invention configured as a bipolar electrode.

FIG. 27 is a schematic diagram of the handheld a probe 20 of FIG. 26 configured as a unipolar electrode.

FIG. 28 is a schematic diagram of the handheld a probe 20 of FIG. 26 configured with both electrodes on the side of the probe.

FIG. 29 is a schematic diagram depicting the use of an ultrasound transducer to determine the thickness of the target tissue 2 around which jaws 40 of the probe of FIG. 19 are placed.

FIG. 30 is a schematic diagram depicting the method as in FIG. 29 wherein the ultrasound transducer is left in place provide an image that allows visual monitoring as the lesion is made.

FIG. 31 is a schematic diagram depicting another embodiment of probe 20 in which electrodes 3, 4 are on a disposable member that fits over a (optionally, hand held) ultrasound probe which may be inserted through a cannula 44.

FIG. 32 is a schematic diagram of the probe 20 of FIG. 31 in which unipolar electrode 11 or bipolar electrodes 3, 4 have points at their ends and can be advanced through a channel in which they reside in the cannula into the tissue under ultrasound guidance.

FIG. 33 is a schematic diagram of the probe 20 of FIG. 31 showing placement of the probe 20 through the central lumen of a scope to be applied non-invasively using a thoracoscopic approach.

FIG. 34 is a schematic diagram depicting the positioning of a therapeutic EMB treatment probe 20 comprising an expandable electrode-bearing balloon 27 according to another embodiment of the present invention inside a blood vessel 401 in the human body.

FIG. 35 is a schematic diagram depicting the positioning of a therapeutic EMB treatment probe 20 comprising an electrode-bearing expandable balloon 27 according to another embodiment of the present invention inside a blood vessel 401 in the human body.

FIG. 36 is a schematic diagram depicting the positioning of a therapeutic EMB treatment probe 20 comprising an insulating sheath 23 bearing electrode 4 according to another embodiment of the present invention inside a blood vessel 401 in the human body.

FIG. 37 is a composite (A & B) schematic diagram depicting the positioning of a therapeutic EMB treatment probe 20 comprising an inflatable stent 19 according to another embodiment of the present invention inside a blood vessel 401 in the human body.

FIG. 38 is a schematic diagram depicting the positioning of a stent 19 left by EMB treatment probe 20 inside a blood vessel 401 in the human body.

FIG. 39 is a schematic diagram of a configuration of probes 20 according to yet another embodiment of the present invention in which one of electrodes 3, 4 is configured as a unipolar electrode with a remote indifferent electrode as a ground.

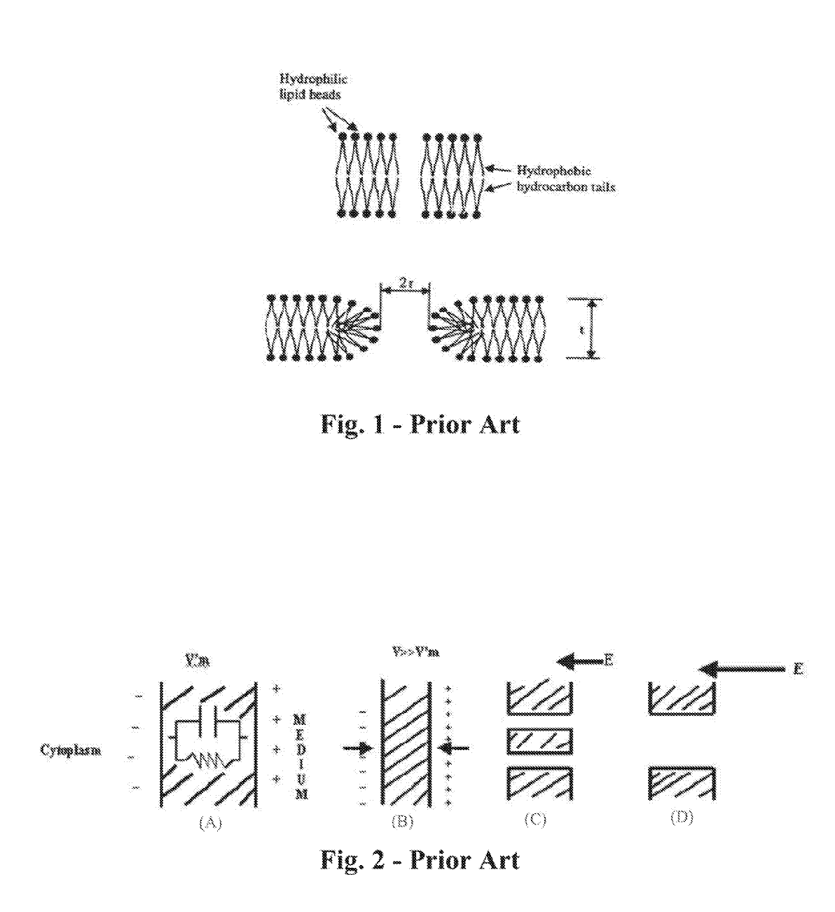

### DETAILED DESCRIPTION

Radiofrequency electrical membrane breakdown (RFEMB or EMB) is a non-thermal method of cell ablation with certain advantages over IRE. EMB causes the immediate destruction of the target cell membrane, such that changes to the cell are immediate and permanent. This mechanism therefore allows immediate determination, using impedance measurements and or measurements of intracellular contents, such a potassium and or uric acid, to indicate the efficacy of the completed treatment. In addition, RFEMB does not cause muscular contraction, allowing the procedure to be carried out under local anesthesia without neuromuscular blockade.

The present invention provides methods and apparatuses for treating atrial fibrillation and other arrhythmias.

In addition, the present invention provides methods and apparatuses for neuromodulation using RFEMB. Such neuromodulation can, for example, effectuate action potential blockade or attenuation, changes in cytokine up-regulation, and other conditions in target neural fibers. In some patients, when the neuromodulatory methods and apparatus of the present invention are applied to renal nerves and/or other neural fibers that contribute to renal neural functions, the neuromodulatory effects induced by the neuromodulation can result in increased urine output, decreased plasma renin levels, decreased tissue (e.g., kidney) and/or urine catecholamines (e.g., norepinephrine), increased urinary sodium excretion, and/or controlled blood pressure. Furthermore, these or other changes can help prevent or treat congestive heart failure, hypertension, acute myocardial infarction, end-stage renal disease, contrast nephropathy, other renal system diseases, and/or other renal or cardio-renal anomalies. The methods and apparatus described herein can be used to modulate efferent or afferent nerve signals, as well as combinations of efferent and afferent nerve signals.

Renal neuromodulation preferably is performed in a bilateral fashion, such that neural fibers contributing to renal function of both the right and left kidneys are modulated. Bilateral renal neuromodulation can provide enhanced therapeutic effect in some patients as compared to renal neuromodulation performed unilaterally, i.e., as compared to renal neuromodulation performed on neural tissue innervating a single kidney. In some embodiments, concurrent modulation of neural fibers that contribute to both right and left renal function may be achieved. In additional or alternative embodiments, such modulation of the right and left neural fibers may be sequential. Bilateral renal neuromodulation may be continuous or intermittent, as desired, by the physician.

The human renal anatomy, including the kidneys, is supplied with oxygenated blood by renal arteries which are connected to the heart by the abdominal aorta. Deoxygenated blood flows from the kidneys to the heart via renal veins (RV) and the inferior vena cava (IVC). More specifically, the renal anatomy also includes renal nerves extending longitudinally along the lengthwise dimension of renal artery (RA) generally within the adventitia of the artery. The renal artery has smooth muscle cells (SMC) that surround the arterial circumference and spiral around the angular axis of the artery. The smooth muscle cells of the renal artery accordingly have a lengthwise or longer dimension extending transverse (i.e., non-parallel) to the lengthwise dimension of the renal artery. The misalignment of the lengthwise dimensions of the renal nerves and the smooth muscle cells is medically defined as “cellular misalignment.”

The cellular misalignment of the renal nerves and the smooth muscle cells may be exploited to selectively affect renal nerve cells with reduced effect on smooth muscle cells. More specifically, because larger cells require a lower electric field strength to exceed the cell membrane's integrity threshold or energy for RFEMB, embodiments of electrodes of the present invention may be configured to align at least a portion of an electric field generated by the electrodes with or near the longer dimensions of the cells to be affected. In specific embodiments, the device has electrodes configured to create an electrical field aligned with or near the lengthwise dimension of the renal artery RA to affect renal nerves. By aligning an electric field so that the field preferentially aligns with the lengthwise aspect of the cell rather than the diametric or radial aspect of the cell, lower field strengths may be used to affect target neural cells, e.g., to break down the neural cell membrane. This is expected to reduce total energy delivered to the system and to mitigate effects on non-target cells in the electric field.

Similarly, the lengthwise or longer dimensions of tissues overlying or underlying the target nerve are orthogonal or otherwise off-axis (e.g., transverse) with respect to the longer dimensions of the nerve cells. Thus, in addition to aligning a pulsed electric field (PEF) with the lengthwise or longer dimensions of the target cells, the PEF may propagate along the lateral or shorter dimensions of the non-target cells (i.e., such that the PEF propagates at least partially out of alignment with non-target smooth muscle cells SMC). Therefore, applying a PEF with propagation lines generally aligned with the longitudinal dimension of the renal artery will preferentially cause EMB in cells of the target renal nerves without unduly affecting the non-target arterial smooth muscle cells SMC.

It will be understood that the RFEMB treatment can be applied from an open operative approach, a minimally invasive laparoscopic approach, or in a percutaneous catheter approach each of which will have different embodiments to accomplish the RFEMB treatment.

In general, the software-hardware controller unit (SHCU) operating the proprietary atrial fibrillation treatment system software according to the present invention facilitates the treatment of an area of cardiac tissue by directing the placement of EMB treatment probe(s) 20, and by delivering electric pulses designed to cause EMB within the targeted tissue to EMB treatment probe(s) 20, all while the entire process may be monitored in real time via one or more two- or three-dimensional imaging devices. The system is such that the treatment may be performed by a physician under the guidance of the software, or may be performed completely automatically, from the process of imaging the treatment area to the process of placing one or more probes using robotic arms operatively connected to the SHCU to the process of delivering electric pulses and monitoring the results of same. Specific components of the invention will now be described in greater detail.

EMB Pulse Generator 16

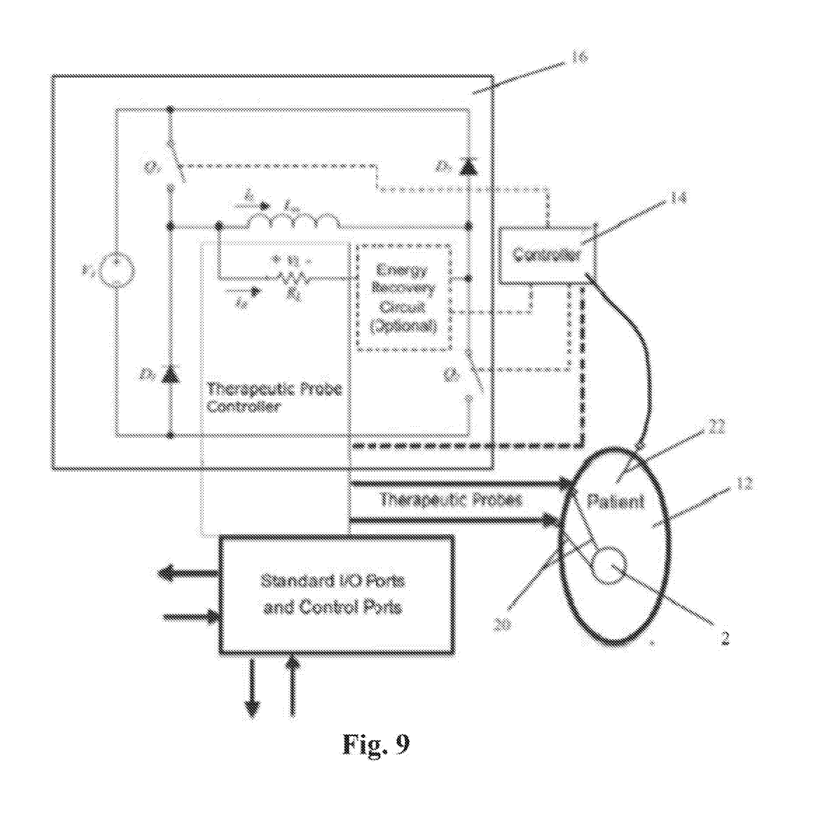

FIG. 9 is a schematic diagram of a system for generation of the electric field necessary to induce EMB of cells 2 within a patient 12. The system includes the EMB pulse generator 16 operatively coupled to Software Hardware Control Unit (SHCU) 14 for controlling generation and delivery to the EMB treatment probes 20 (two are shown) of the electrical pulses necessary to generate an appropriate electric field to achieve EMB. FIG. 9 also depicts optional onboard controller 15 which is preferably the point of interface between EMB pulse generator 16 and SHCU 14. Thus, onboard controller 15 may perform functions such as accepting triggering data from SHCU 14 for relay to pulse generator 16 and providing feedback to SHCU regarding the functioning of the pulse generator 16. The EMB treatment probes 20 (described in greater detail below) are placed in proximity to the soft tissue cells 2 which are intended to be ablated through the process of EMB and the bipolar pulses are shaped, designed and applied to achieve that result in an optimal fashion. A temperature probe 22 may be provided for percutaneous temperature measurement and feedback to the controller of the temperature at, on or near the electrodes. The controller may preferably include an onboard digital processor and a memory and may be a general purpose computer system, programmable logic controller or similar digital logic control device. The controller is preferably configured to control the signal output characteristics of the signal generation including the voltage, frequency, shape, polarity and duration of pulses as well as the total number of pulses delivered in a pulse train and the duration of the inter pulse burst interval.

With continued reference to FIG. 9, the EMB protocol calls for a series of short and intense bi-polar electric pulses delivered from the pulse generator through one or more EMB treatment probes 20 inserted directly into, or placed around the target tissue 2. The bi-polar pulses generate an oscillating electric field between the electrodes that induce a similarly rapid and oscillating buildup of transmembrane potential across the cell membrane. The built up charge applies an oscillating and flexing force to the cellular membrane which upon reaching a critical value causes rupture of the membrane and spillage of the cellular content. Bipolar pulses are more lethal than monopolar pulses because the pulsed electric field causes movement of charged molecules in the cell membrane and reversal in the orientation or polarity of the electric field causes a corresponding change in the direction of movement of the charged molecules and of the forces acting on the cell. The added stresses that are placed on the cell membrane by alternating changes in the movement of charged molecules create additional internal and external changes that cause indentations, crevasses, rifts and irregular sudden tears in the cell membrane causing more extensive, diverse and random damage, and disintegration of the cell membrane.

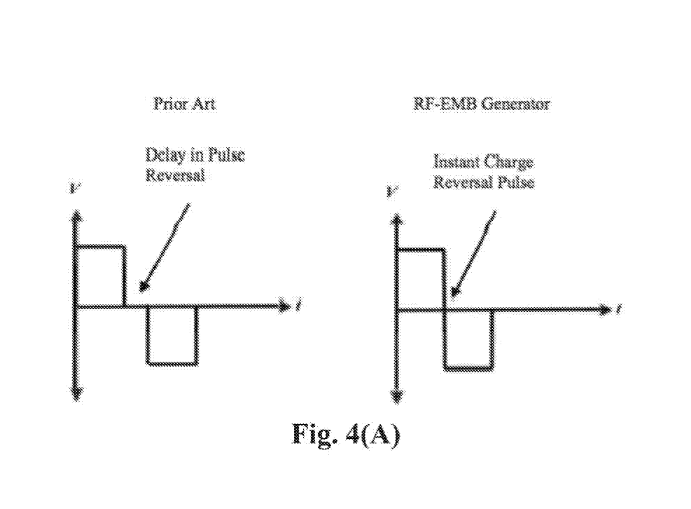

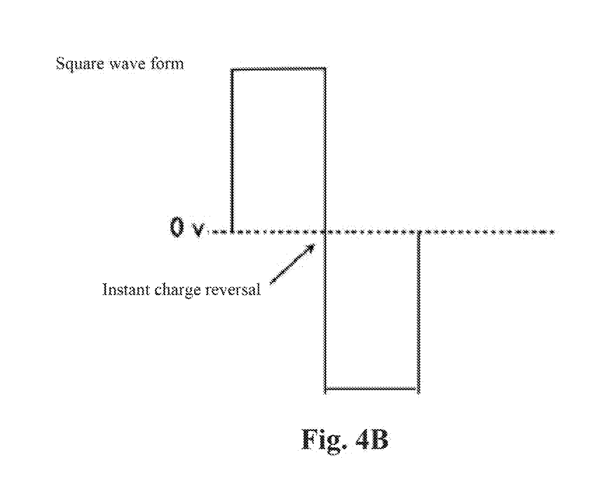

With reference to FIG. 4B, in addition to being bi-polar, the preferred embodiment of electric pulses is one for which the voltage over time traces a square wave form and is characterized by instant charge reversal pulses (ICR). A square voltage wave form is one that maintains a substantially constant voltage of not less than 80% of peak voltage for the duration of the single polarity portion of the trace, except during the polarity transition. An instant charge reversal pulse is a pulse that is specifically designed to ensure that substantially no relaxation time is permitted between the positive and negative polarities of the bi-polar pulse (See FIG. 4A). That is, the polarity transition happens virtually instantaneously.

The destruction of dielectric cell membranes through the process of Electrical Membrane Breakdown is significantly more effective if the applied voltage pulse can transition from a positive to a negative polarity without delay in between. Instant charge reversal prevents rearrangement of induced surface charges resulting in a short state of tension and transient mechanical forces in the cells, the effects of which are amplified by large and abrupt force reversals. Alternating stress on the target cell that causes structural fatigue is thought to reduce the critical electric field strength required for EMB. The added structural fatigue inside and along the cell membrane results in or contributes to physical changes in the structure of the cell. These physical changes and defects appear in response to the force applied with the oscillating EMB protocol and approach dielectric membrane breakdown as the membrane position shifts in response to the oscillation, up to the point of total membrane rupture and catastrophic discharge. This can be analogized to fatigue or weakening of a material caused by progressive and localized structural damage that occurs when a material is subjected to cyclic loading, such as for example a metal paper clip that is subjected to repeated bending. The nominal maximum stress values that cause such damage may be much less than the strength of the material under ordinary conditions. The effectiveness of this waveform compared to other pulse waveforms can save up to ⅕ or ⅙ of the total energy requirement.

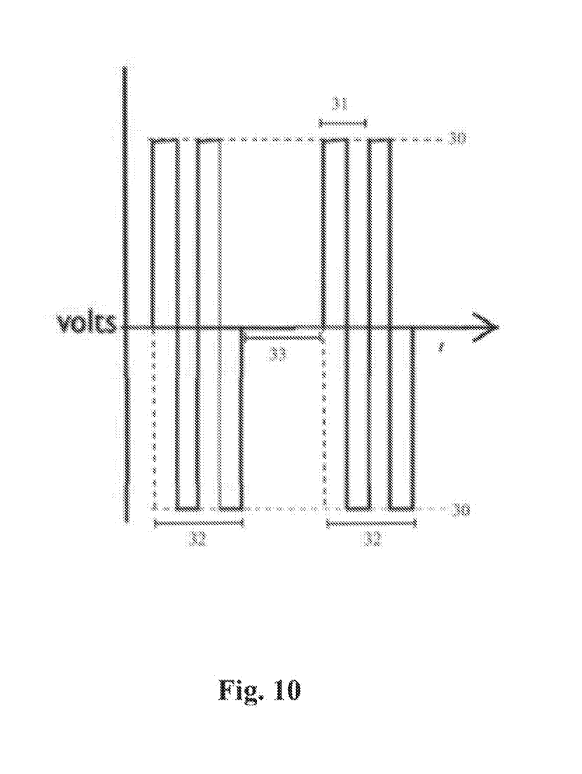

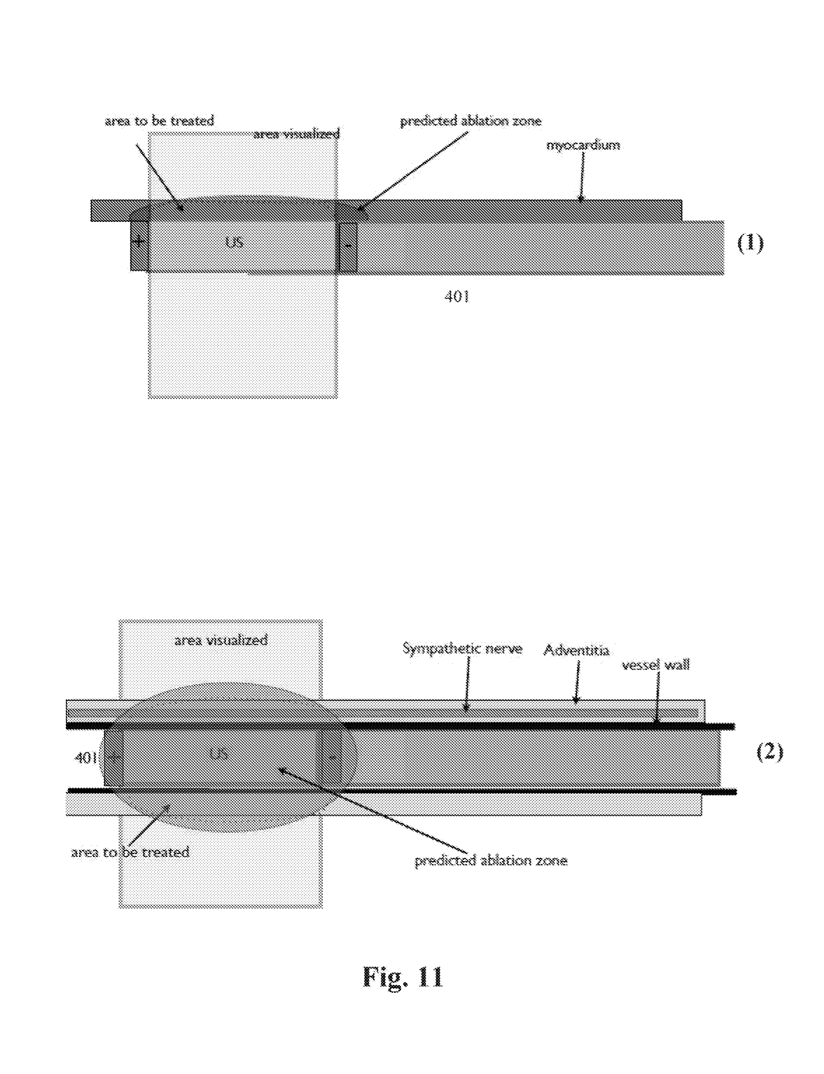

With reference to FIG. 10, another important characteristic of the applied electric field is the field strength (Volts/cm) which is a function of both the voltage 30 applied to the electrodes by the pulse generator 16 and the electrode spacing. Typical electrode spacing for a bi-polar probe might be 1 cm, while spacing between multiple electrodes can be selected by the surgeon and might typically be from 0.75 cm to 1.5 cm. A pulse generator for application of the present invention is capable of delivering up to a 10 kV potential. The actual applied field strength will vary over the course of a treatment to control circuit amperage which is the controlling factor in heat generation, and patient safety (preventing large unanticipated current flows as the tissue impedance falls during a treatment). Where voltage and thus field strength is limited by heating concerns, the duration of the treatment cycle may be extended to compensate for the diminished charge accumulation. Absent thermal considerations, a preferred field strength for EMB is in the range of 1,500 V/cm to 10,000 V/cm.

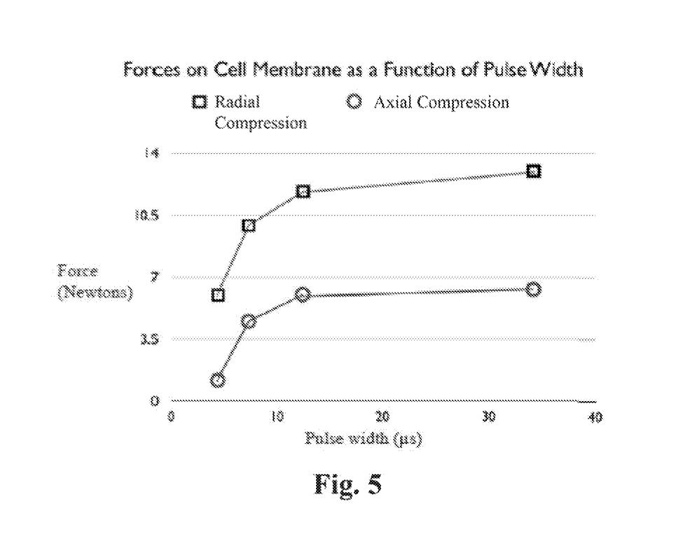

With continued reference to FIG. 10, the frequency 31 of the electric signal supplied to the EMB treatment probes 20, and thus of the field polarity oscillations of the resulting electric field, influences the total energy imparted on the subject tissue and thus the efficacy of the treatment but are less critical than other characteristics. A preferred signal frequency is from 14.2 kHz to less than 500 kHz. The lower frequency bound imparts the maximum energy per cycle below which no further incremental energy deposition is achieved. With reference to FIG. 5, the upper frequency limit is set based on the observation that above 500 kHz, the polarity oscillations are too short to develop enough motive force on the cell membrane to induce the desired cell membrane distortion and movement. More specifically, at 500 kHz the duration of a single full cycle is 2 μs of which half is of positive polarity and half negative. When the duration of a single polarity approaches 1 μs there is insufficient time for charge to accumulate and motive force to develop on the membrane. Consequently, membrane movement is reduced or eliminated and EMB does not occur. In a more preferred embodiment the signal frequency is from 100 kHz to 450 kHz. Here the lower bound is determined by a desire to avoid the need for anesthesia or neuromuscular-blocking drugs to limit or avoid the muscle contraction stimulating effects of electrical signals applied to the body. The upper bound in this more preferred embodiment is suggested by the frequency of radiofrequency thermal ablation equipment already approved by the FDA, which has been deemed safe for therapeutic use in medical patients.

In addition, the energy profiles that are used to create EMB also avoid potentially serious patient risks from interference with cardiac sinus rhythm, as well as localized barotrauma, which can occur with other therapies.

EMB Treatment Probes 20

EMB treatment probes are comprised of at least one therapeutic probe 20 capable of delivering therapeutic EMB pulsed radio frequency energy or biphasic pulsed electrical energy under sufficient conditions and with sufficient treatment parameters to completely break down the membranes of the targeted cardiac or sympathetic nerve tissue.

In a first preferred embodiment, probes 20 are preferably of the catheter type known in the art and having one or more central lumens to, among other things, allow probe 20 to be placed over a guide wire for ease of insertion and/or placement of probe 20 within a vessel 400 of the human body according to the Seldinger technique. A catheter for this purpose may be an angiographic balloon type catheter of the type known in the art, sized between 5 French to 8 French and made of materials generally used for angiographic catheters, such as silicone or latex, or any other biocompatible, flexible material. Alternatively, and preferably for treatment of the sympathetic nerve, a catheter for this purpose may be an angiographic balloon dilatation catheter.

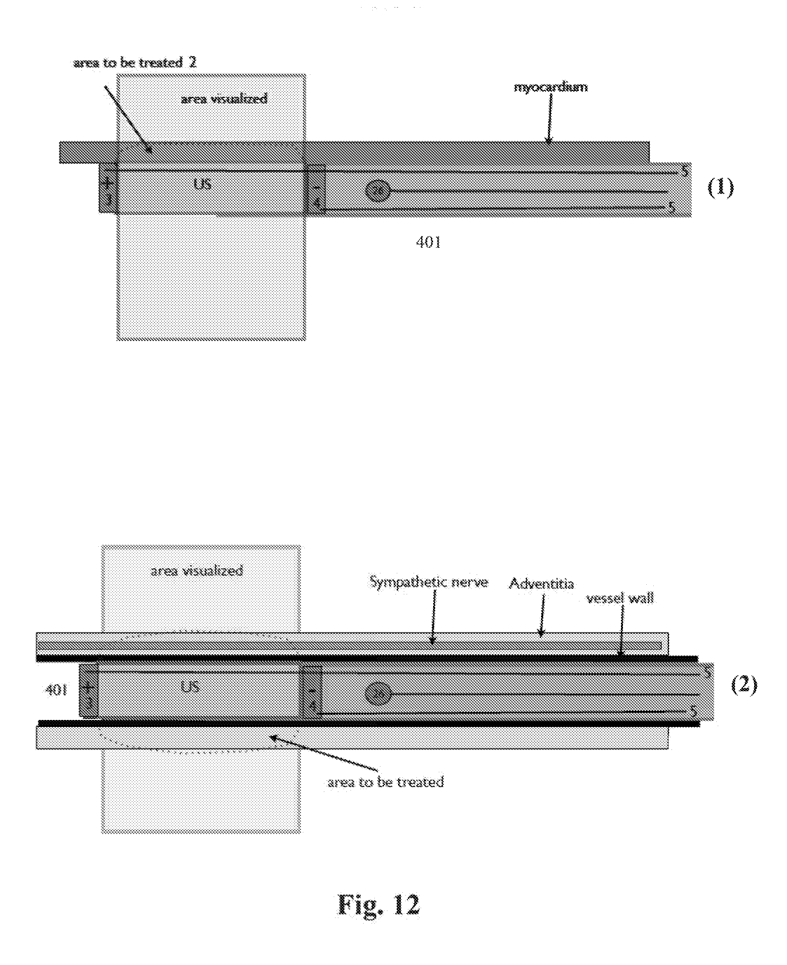

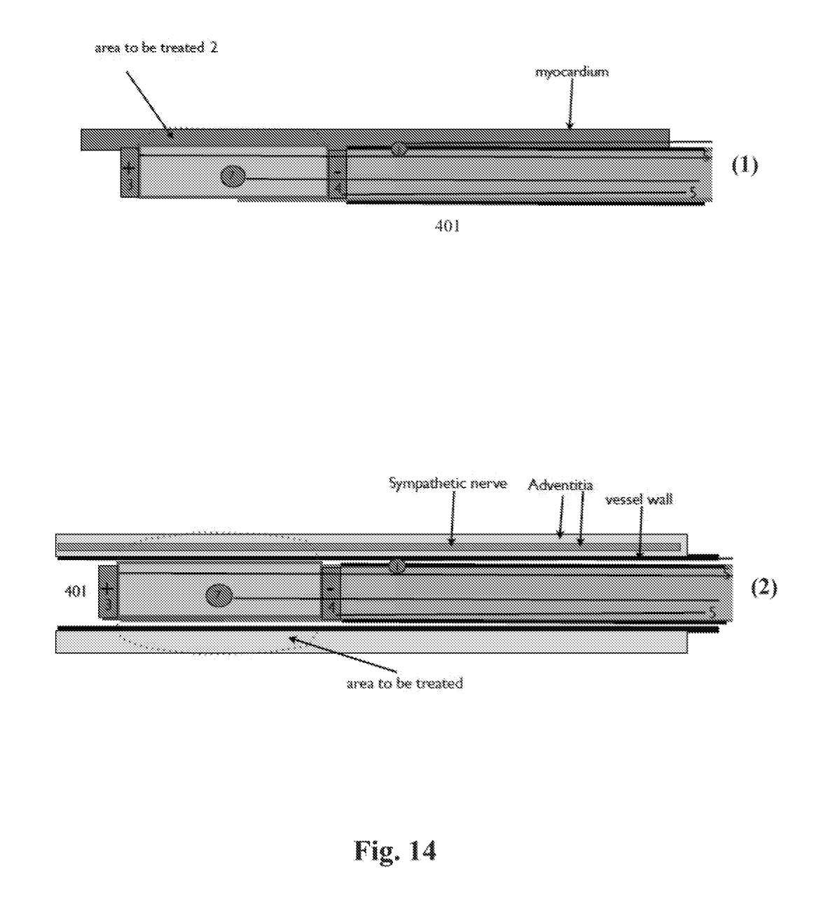

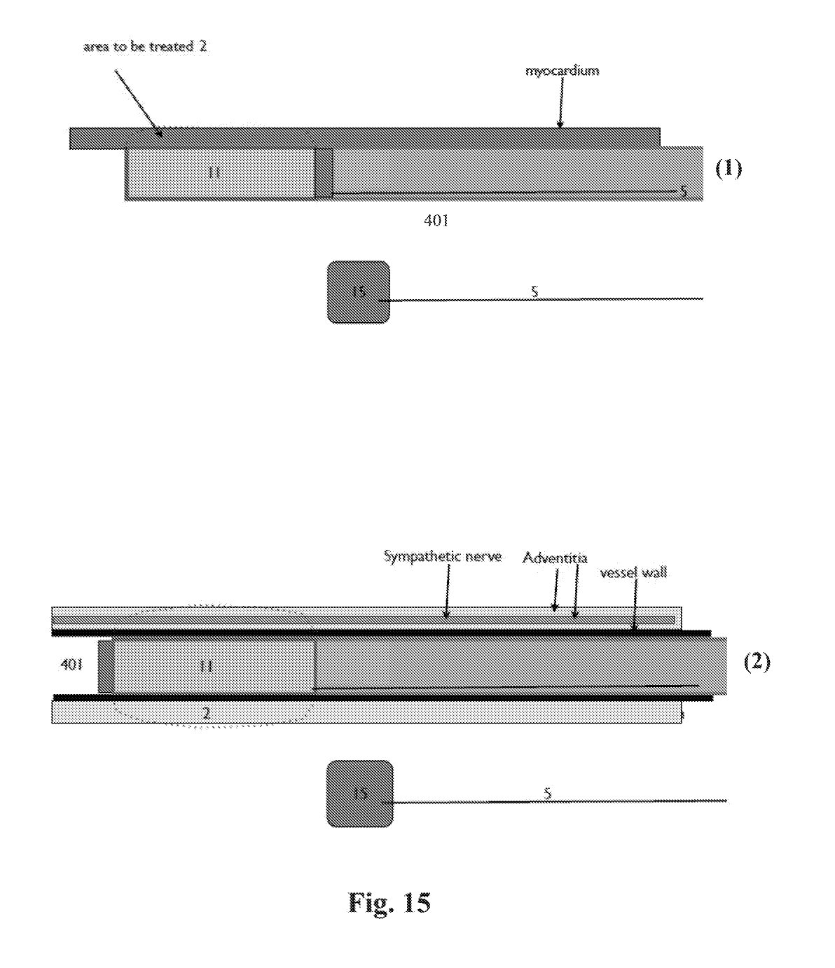

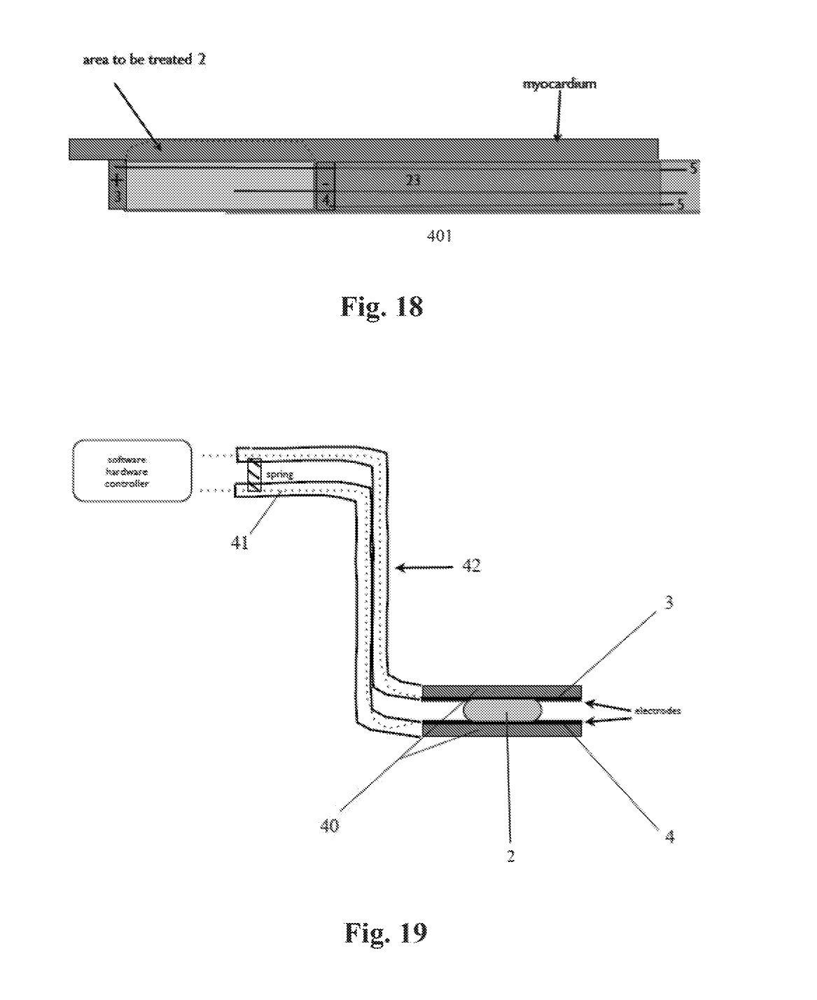

In one preferred embodiment, illustrated in FIGS. 12-14, probe 20 further comprises one positive 3 and one negative 4 electrode disposed on an outer surface of probe 20 and spaced apart by a distance along the longitudinal axis of probe 20 such that current sufficient to deliver the EMB pulses described herein may be generated between the electrodes 3, 4. The spacing between positive 3 and negative 4 electrodes may vary by design preference, wherein a larger distance between electrodes 3, 4 provides a larger treatment area 2. FIGS. 12-14 depict electrodes 3, 4 on an outer surface of probe 20; alternatively, electrodes 3, 4 are integral to the surface of probe 20. In certain embodiments, the area between the electrodes can constitute an ultrasound transducer. In yet another embodiment, as shown in FIG. 18, one of electrodes 3, 4 (negative electrode 4 as shown in FIG. 18) may be placed on the end of an insulated sheath 23 that either partially or fully surrounds probe 20 along a radial axis thereof and is movable along a longitudinal axis of probe 20 relative to the tip thereof (on which positive electrode 3 is located as shown in FIG. 18) to provide even further customizability with respect to the distance between electrodes 3, 4 and thus the size of treatment area 2. Insulating sheath 23 is preferably made of an inert material compatible with bodily tissue, such as Teflon® or Mylar®. One means for enabling the relative movement between probe 20 and insulating sheath 23 is to attach insulating sheath 23 to a fixed member (i.e., a handle) at a distal end of probe 20 opposite the tip of probe 20 by a screw mechanism, the turning of which would advance and retract the insulating sheath 23 along the body of the probe 20. Other means for achieving this functionality of EMB treatment probe 20 are known in the art.

Without limitation, electrodes may be flat (i.e., formed on only a single side of probe 20), cylindrical and surrounding probe 20 around an axis thereof, etc. Electrodes 3, 4 are made of an electrically conductive material. Electrodes 3, 4 may be operatively connected to EMB pulse generator 16 via one or more insulated wires 5 for the delivery of EMB pulses from generator 16 to the treatment area 2. Connection wires 5 may either be intraluminal to the catheter probe 20 or extra-luminal on the surface of catheter probe 20.

Also in a preferred embodiment, as shown in FIG. 12, probe 20 further comprises an electromagnetic (EM) sensor/transmitter 26 that allows visual location of probe 20 within the patient relative to the 3D Fused Image of the treatment area (described in further detail below). EM sensors 26 may be located on both probe 20 and optional insulating sheath 23 to send information to the Software Hardware Controller Unit (SHCU) for determining the positions and/or relative positions of these two elements and thus the size of the treatment area, preferably in real time. EM sensors 26 may be a passive EM tracking sensor/field generator, such as the EM tracking sensor manufactured by Traxtal Inc. Alternatively, instead of utilizing EM sensors, EMB treatment probes 20 may be tracked in real time and guided using endoscopy, ultrasound or other imaging means known in the art.

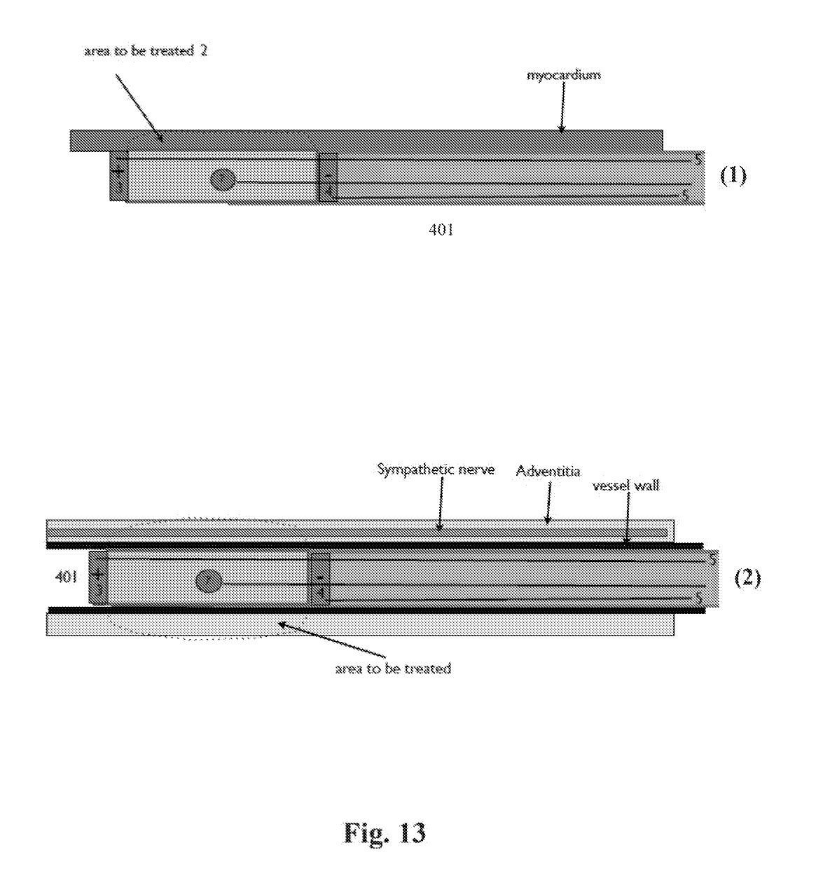

Also in a preferred embodiment, as shown in FIG. 13, probe 20 further comprises a thermocouple 7 on the insulating surface thereof such that the temperature at the wall of the catheter can be monitored and the energy delivery to electrodes 3, 4 modified to maintain a desired temperature at the wall of the probe 20 as described in further detail above. Thermocouple 7 may be, i.e., a Type K-40AWG thermocouple with Polyimide Primary/Nylon Bond Coat insulation and a temperature range of −40 to +180 C, manufactured by Measurement Specialties.

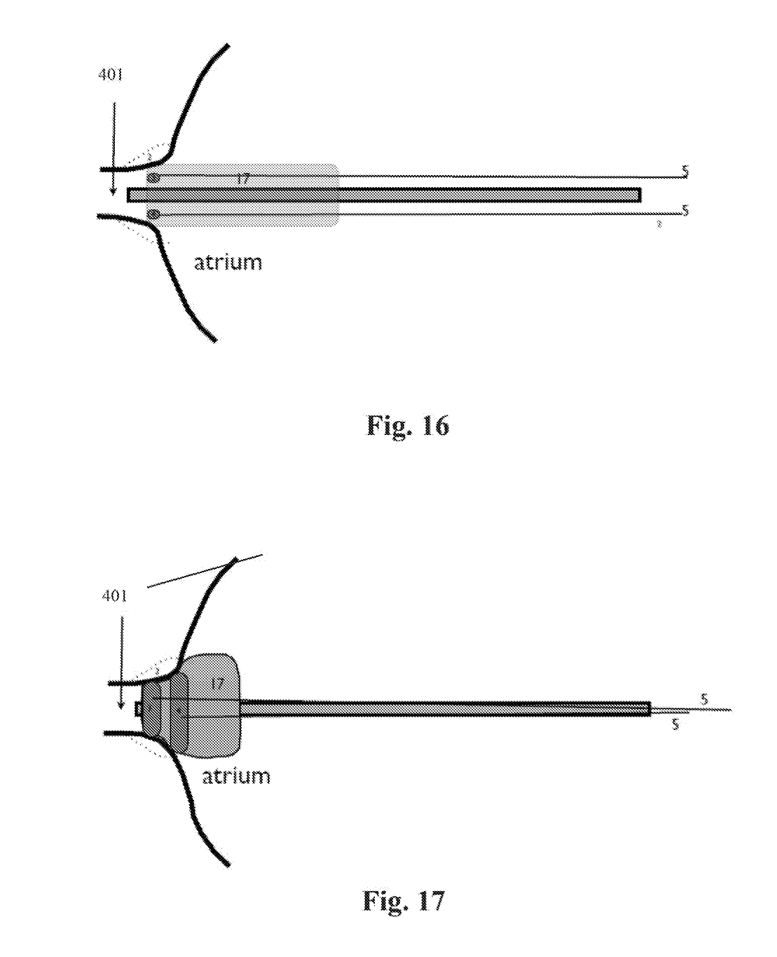

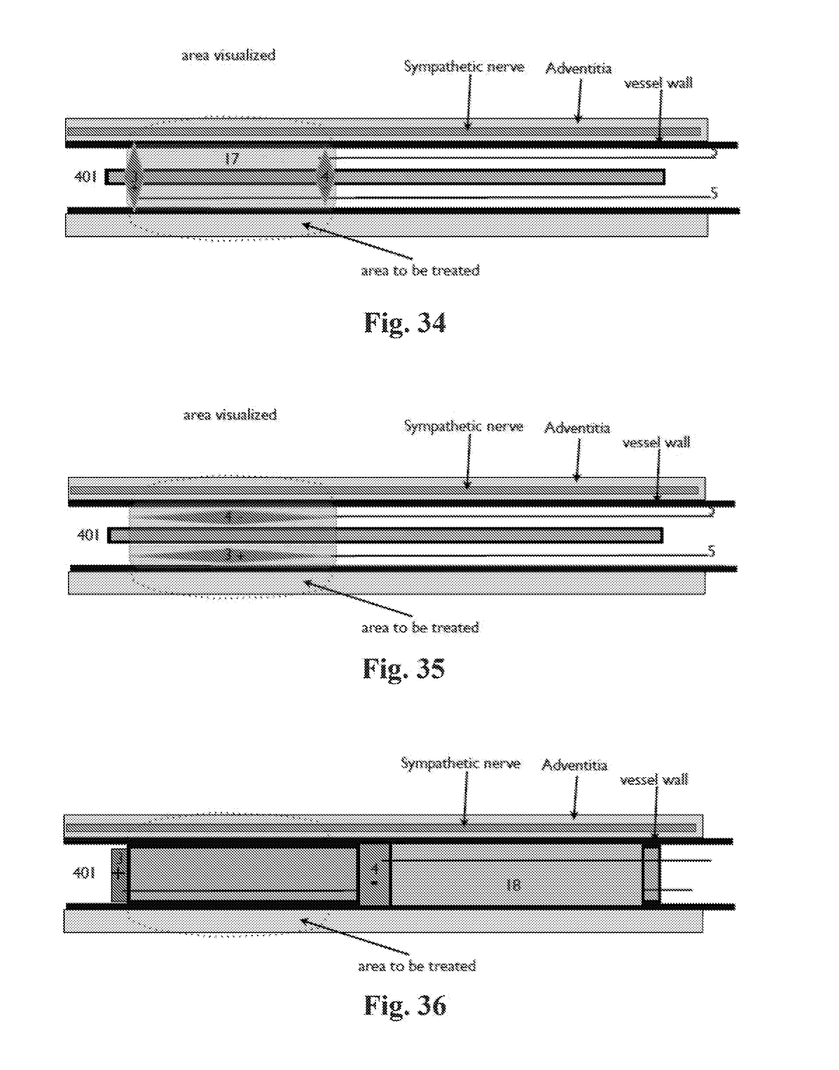

In yet another alternative embodiment of EMB treatment probes 20, unipolar or bipolar electrodes are placed on an expandable balloon 17, the inflation of which may be controlled by the SHCU via a pneumatic motor or air pump, etc. In this embodiment, when the balloon 17 is placed inside a the orifice of the pulmonary vein or blood vessel 401 in the human body (proximate a designated treatment area) and inflated, the electrodes on the balloon's surface are forced against the wall of the blood vessel 401 to provide a path for current to flow between the positive and negative electrodes (see FIGS. 16 and 34). The positive and negative electrodes can have different configurations on the balloon 17, i.e., they may be arranged horizontally around the circumference of the balloon 17 as in FIGS. 16 and 34, or longitudinally along the long axis of the balloon as in FIGS. 17 and 35. In some embodiments, more than one each of positive and negative electrodes may be arranged on a single balloon.

In certain embodiments, such as for the treatment of atrial fibrillation and arrhythmias, the catheter-type EMB probe 20 can have a coil of wire proximate to its distal end. Current placed through this wire coil makes the wire coil into an electromagnet. While the electromagnet is activated, a strong external magnet may be positioned outside of the patient such that the catheter-type EMB probe 20 is held against the myocardium in the area of the treatment by the magnetic force. In this way, the EMB probe 20 is held in place during the treatment.

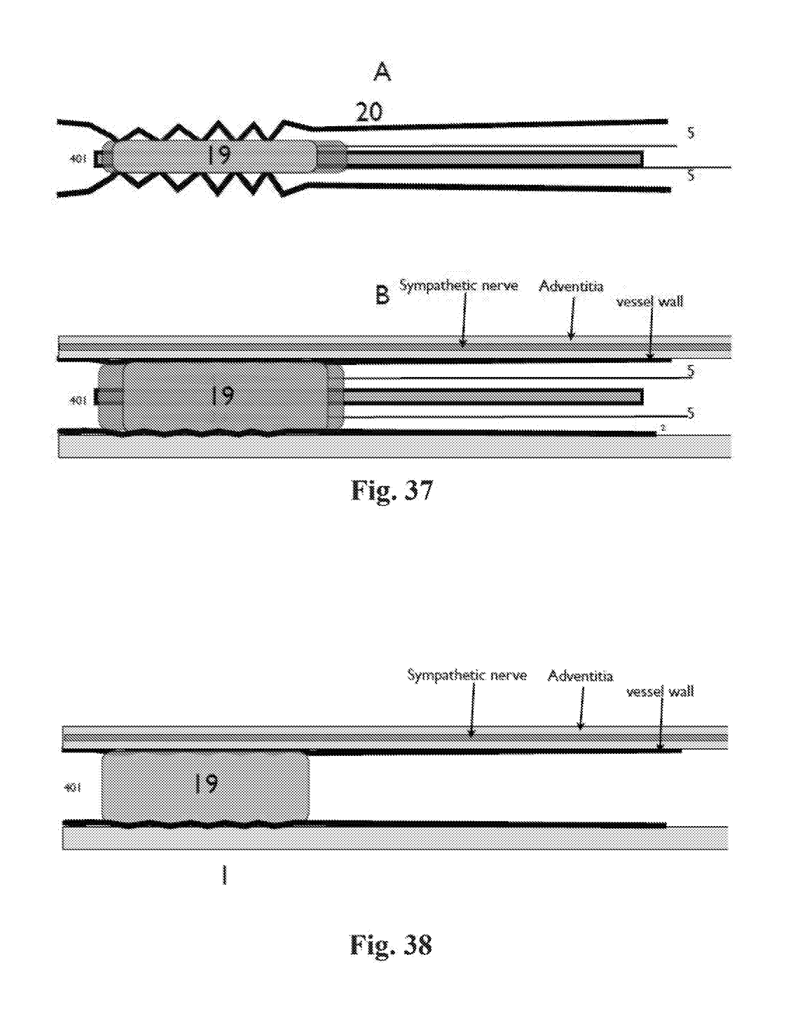

It is not uncommon for patients who need therapy for renal neuromodulation to also require supportive vascular therapy for atherosclerosis in the vascular region where the neuromodulation procedure is focused, in order to enhance the safety and effectiveness of such therapy. Therefore, in yet another embodiment, EMB catheter-type probe 20 could deliver a stent 19 to the abnormal region in the renal blood vessel which is associated with a narrowing causing obstruction. This configuration would allow the delivery of an EMB treatment protocol at the same time as stent 19 is used to expand a stricture in a vessel, making the overall therapy more effective. Stent 19 may also comprise conducting and non-conducting areas which correspond to the unipolar or bipolar electrodes on EMB probe 20 (or, for a unipolar electrode, the stent would be made of an electrically conducting material which will couple with the electrode on the balloon catheter). An example treatment protocol would include placement of EMB probe 20 having balloon 17 with a stent 19 over the balloon 17 in its non expanded state (FIG. 37(A)), expansion of balloon 17 which in turn expands stent 19 (FIG. 37(B)), delivery of the RFEMB treatment, and removal of the EMB treatment probe 20 and balloon 17, leaving stent 19 in place in the patient (see FIG. 38).

In another embodiment, interior lumen 10 may be sized to allow for the injection of biochemical or biophysical nano-materials there through into the EMB lesion to enhance the efficacy of the local ablative effect, or the effect of the EMB treatment, or to allow injection of reparative growth stimulating drugs, chemicals or materials. An interior lumen 10 of the type described herein may also advantageously allow the collection and removal of tissue or intracellular components from the treatment area or nearby vicinity, for any desired testing. This functionality can be used for such purposes before, during or after the application of EMB pulses from the EMB treatment probe 20.

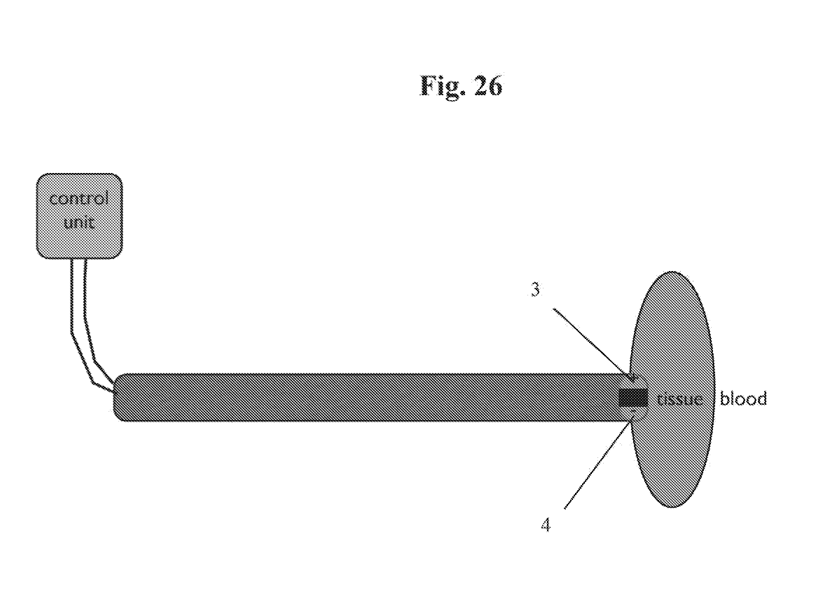

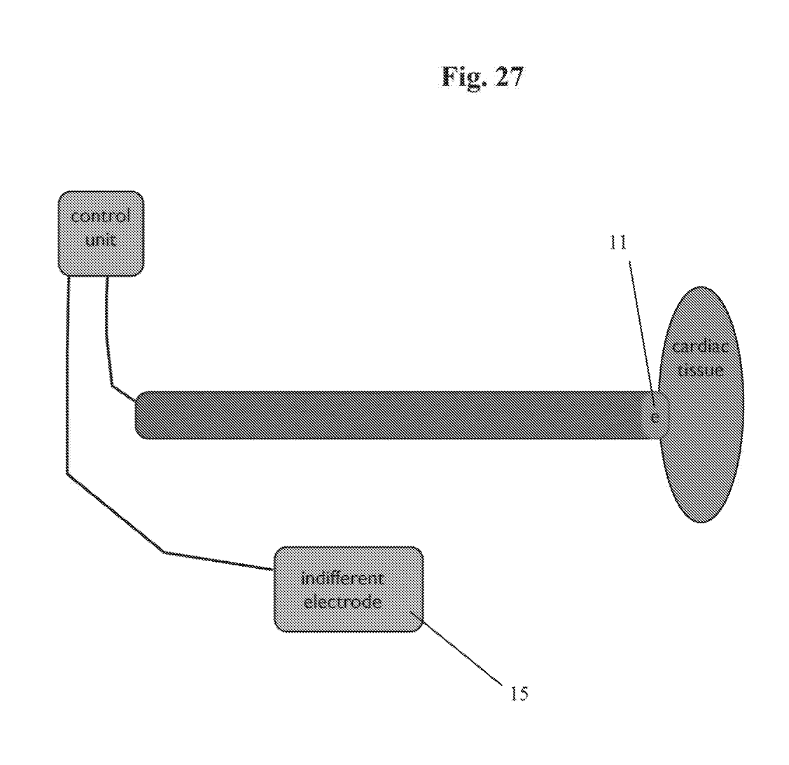

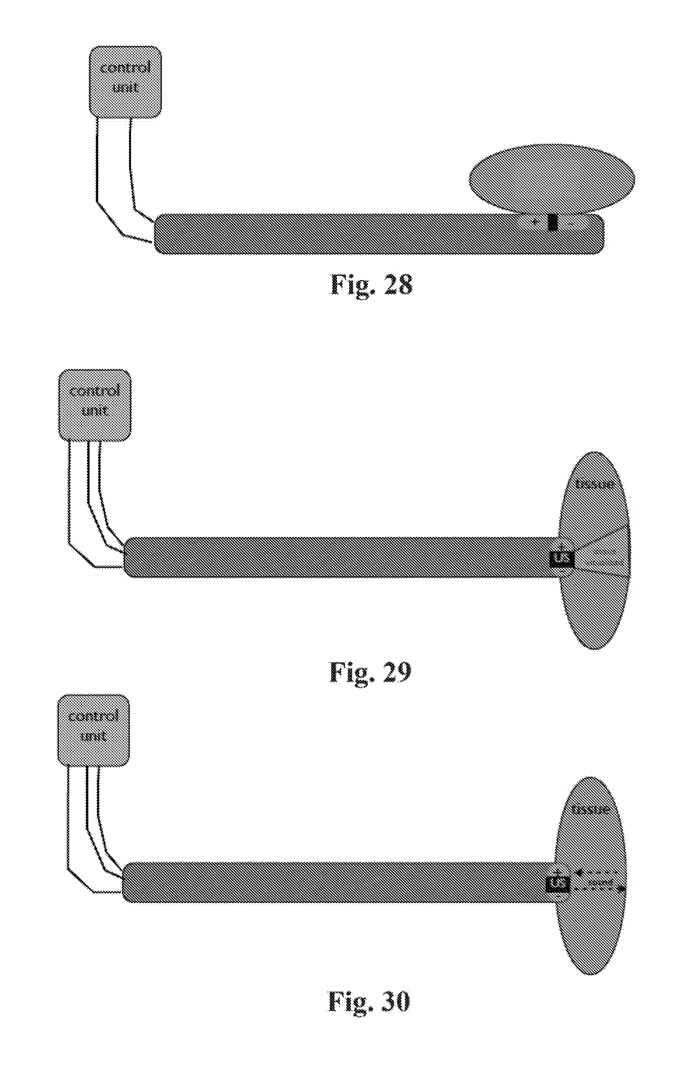

FIGS. 26 through 29 illustrate handheld embodiments of probe 20 when configured as a bipolar electrode 3, 4 (FIG. 26) or as a unipolar electrode 11 (FIG. 27) with a remote indifferent electrode 15 elsewhere on or near the patient's body. In this embodiment, the electrode 11 or electrodes 3, 4 are incorporated into a handheld probe 20 to allow the surgeon to place the active electrode portion of the probe against the surface of the cardiac tissue for delivery of RFEMB treatment. FIG. 28 shows an embodiment of hand held probe 20 in which the electrode 11I or electrodes 3, 4 are on the distal end of the probe 20 but located on a side rather than it end as shown in FIGS. 26-27.

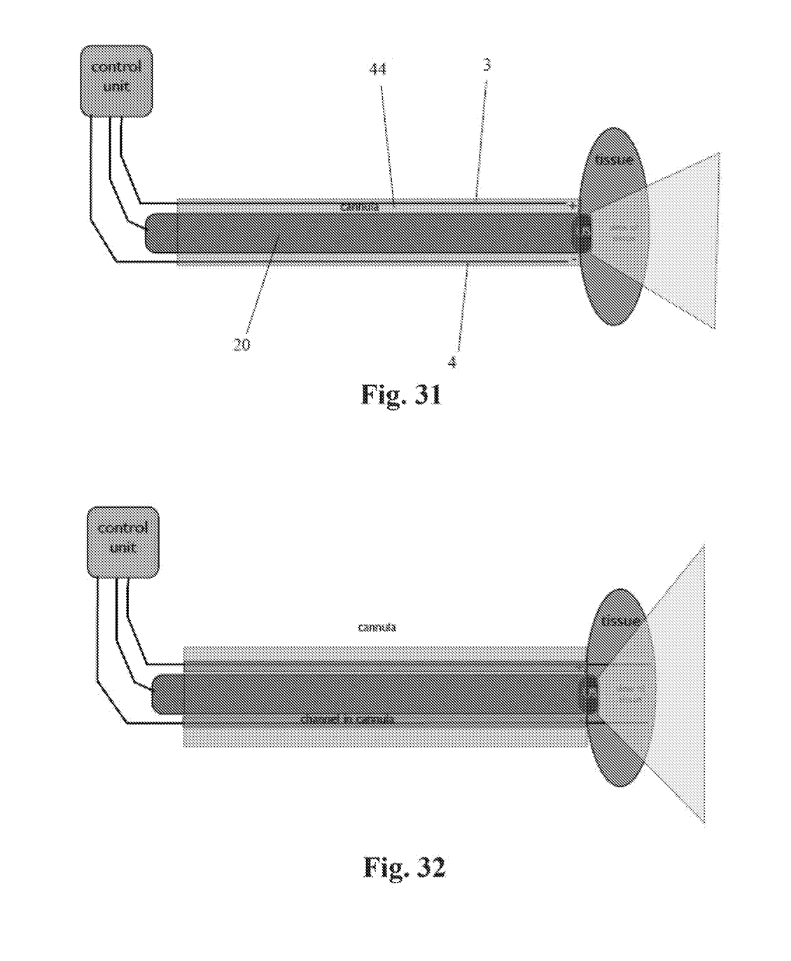

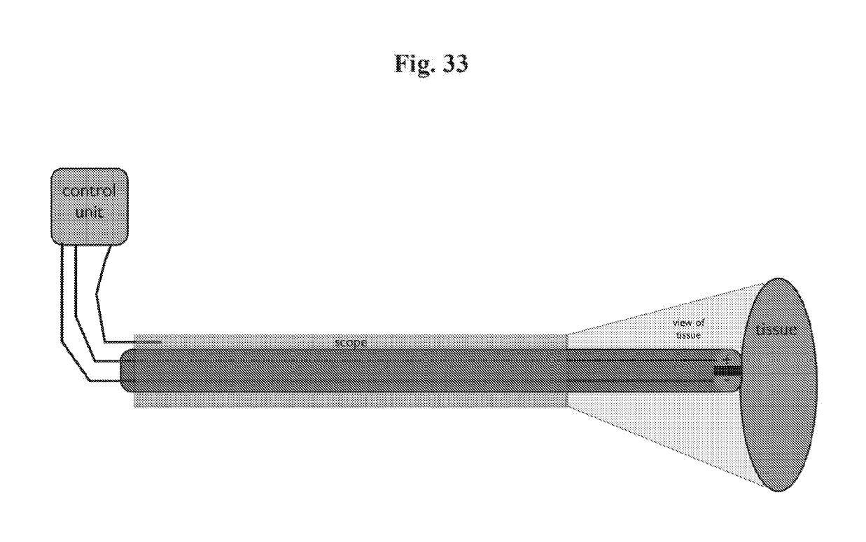

FIGS. 31-33 show another embodiment of probe 20 in which electrodes 3, 4 are on a disposable member that fits over a (optionally, hand held) ultrasound probe which may be inserted through a cannula 44. Optionally, the unipolar electrode 11 or bipolar electrodes 3, 4 can have points at their ends and can be advanced through a channel in which they reside in the cannula into the tissue under ultrasound guidance (see FIG. 32). This handheld probe 20 is preferably of such a length and width to be able to be placed through the central lumen of a scope and applied non-invasively using, where appropriate for the tissue targeted, a thoracoscopic approach (see FIG. 33).

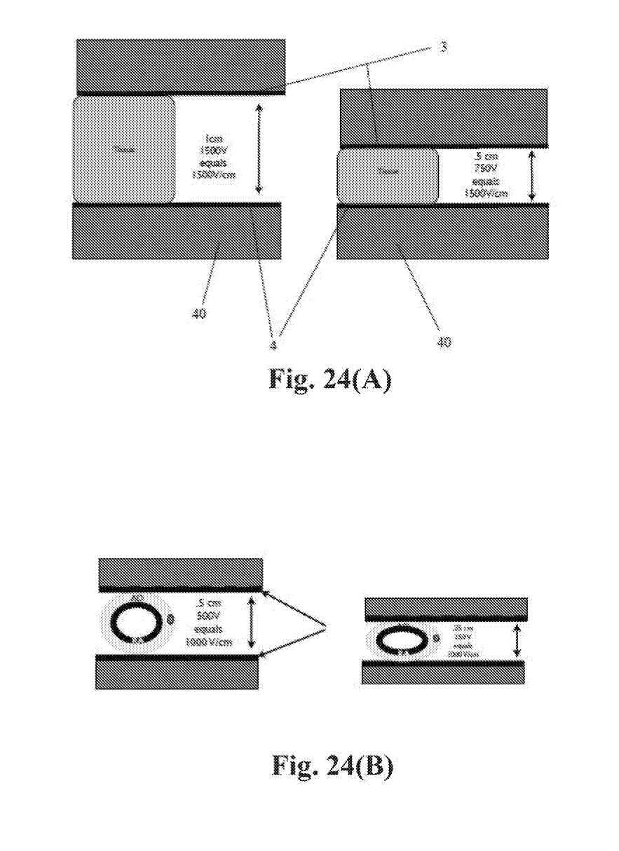

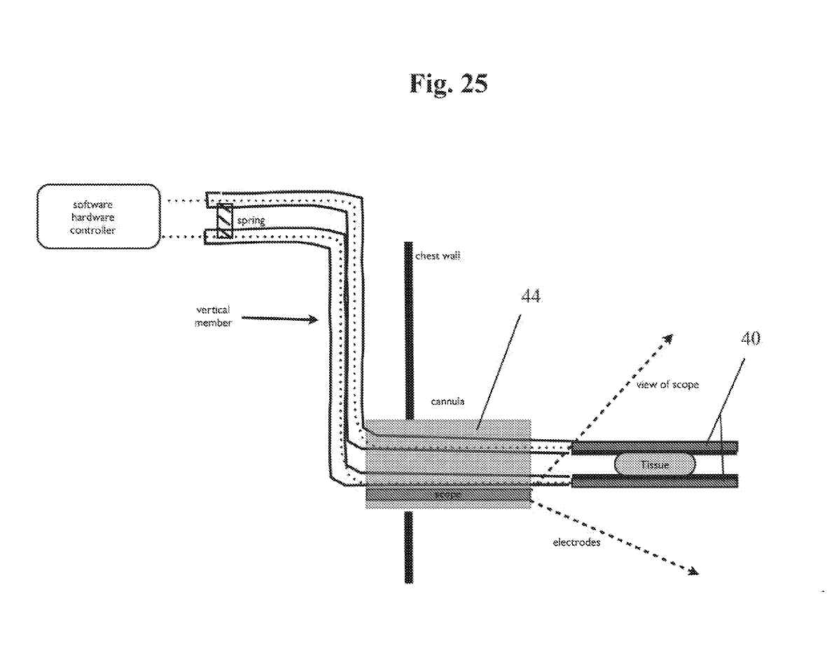

Referring to FIGS. 19 (atrial fibrillation and arrhythmia treatment) and 25 (sympathetic nerve treatment), in another preferred embodiment, probes 20 are specially designed clamps with electrodes attached in various configurations with insulation configured to allow adjustment in electrode exposure and area of EMB pulse contact for tissue ablation. Clamp-type probes 20 comprise positive 3 and negative 4 electrodes on extended, opposing and parallel jaws 40, the jaws 40 being movable relative to one another in an axis perpendicular to their longitudinal plane. Jaws 40 are preferably injection molded from biocompatible materials, or formed by any other means known in the art; one possible clamp for this use is the clamp probe manufactured by Medtronic. Electrodes 3, 4 are placed on the interior surface of each jaw 40 such that electrodes 3, 4 face each other. The jaws are further configured so that the same distance is maintained between the jaws throughout the length of the clamp as the clamp is opened and closed. The clamping probe also preferably comprises a handle member 41 parallel to jaws 40, and a body member 42 perpendicular to handle 41 and jaws 40, jaws 40 and handle 41 being slidably attached to body member 42 along its longitudinal axis. The distance between jaws 40 can be calculated mechanically or electronically through a mechanism placed in the handle 41 (such as a spring as shown in FIGS. 19 and 25) and the various parameters supplied by pulse generator 16 (voltage, pulse number, pulse width, inter-pulse distance, etc.) may be altered based on the calculated distance between electrodes 3, 4 on jaws 40. Jaws 40 may also comprise a sensing mechanism (not shown) to determine the thickness of the target tissue 2 around which jaws 40 are placed. For example, an ultrasonic transducer may be used for this purpose. Because the heart and rental artery are fluid fill structures, the method used could be similar to that used for bladder volume scanning, in which the distance of the path of the sound is calculated by knowing the speed within the tissue and the time it takes for the return signal (see FIGS. 29 and 30). This information may be fed back to the SHCU 14, which in turn may adjust the ablation parameters to adequately ablate the target tissue 2 of the given thickness. In one example, the voltage provided to electrodes 3, 4 may be automatically adjusted to maintain a specified or calculated voltage density based on other parameters of the target tissue 2. For instance, electrodes 3, 4 might be 1 cm apart due to the thickness of the myocardial target tissue 2, and a voltage of 1500 volts applied equates to a voltage density of 1500 volts/cm. In another example the tissue thickness might be 0.5 cm and a voltage of 750 volts applied equates to a voltage density of 1500 volts per cm (see FIG. 24(A)). Alternatively, electrodes 3, 4 might be 0.5 cm apart due to the thickness of the renal artery, and a voltage of 500 volts applied equates to a voltage density of 1000 volts/cm. In another example the tissue thickness might be 25 cm due to compression of the renal artery and a voltage of 250 volts applied equates to a voltage density of 1000 volts per cm (see FIG. 24(B)).

Preferably, a thermocouple 7 can be incorporated into one or both jaws 40 adjacent to electrodes 3, 4 to measure temperature at the treatment site. This temperature reading can feed back to the SHCU 14 and the pulsing characteristics changed to prevent any potential thermal damage to the treatment area 2. Optionally, the ultrasound transducer used for calculating the thickness of the target tissue 2 may also provide an image that allows visual monitoring as the lesion is made (see FIG. 30).

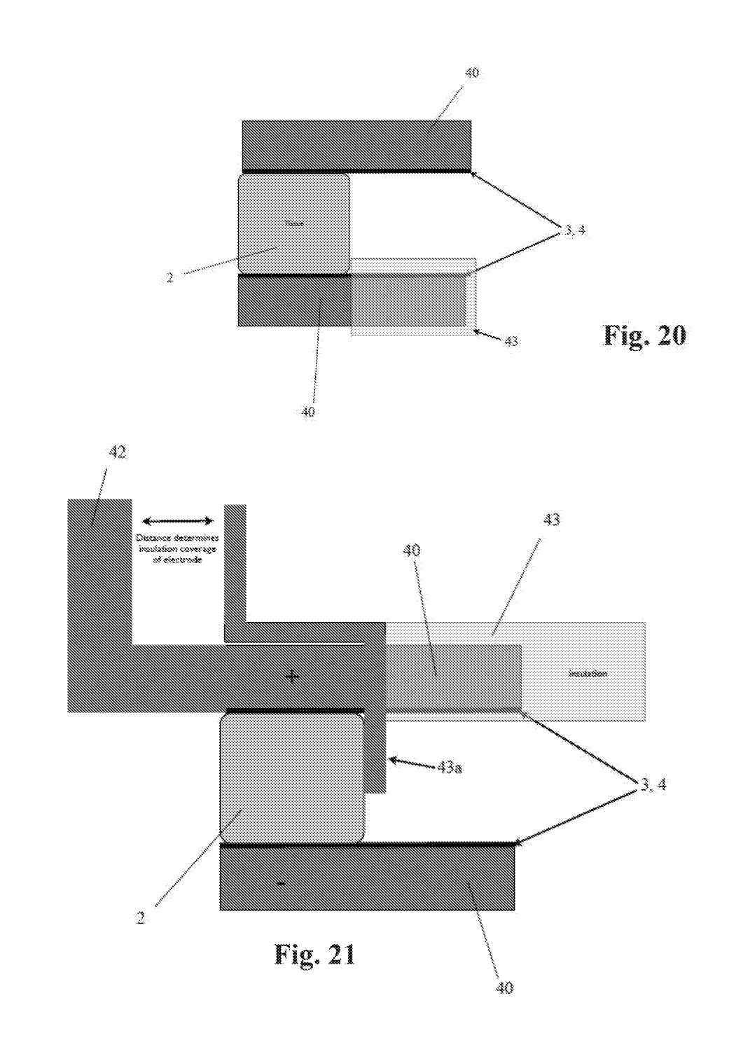

In a preferred embodiment, shown in FIGS. 20 and 21, a portion of one or both jaws 40 and/or electrodes 3, 4 may be covered with an insulating material 43 on an area that will not be in contact with the target tissue 2. Insulating material 43 is preferably made from biocompatible such as silicon or Mylar®. Insulating material 43 may take the form of a sheath that wraps axially around a portion of one or more jaws 40 and electrodes 3, 4, which may be permanently affixed or removable and re-adjustable based on the patient-specific geometry of the treatment area. Insulating material 43 may also take the form of a pocket able to be slipped over a distal end of one or more jaws 40 and electrodes 3, 4. In a bipolar mode where one jaw 40 contains a positive electrode 3 and the other jaw 40 contains a negative electrode 4, only one electrode needs to be insulated to prevent current flow. Where insulating member 43 is permanently affixed to one of the two jaws 40, insulating member 43 may further comprise a perpendicular projection 43a at an open end which prevents insulating member 43 from covering any portion of electrode 3 that is in contact with target tissue 2 as insulating member is slid over jaw 40 and electrode 3 beginning at the distal end of jaw 40 by abutting target tissue 2 (see FIG. 21).

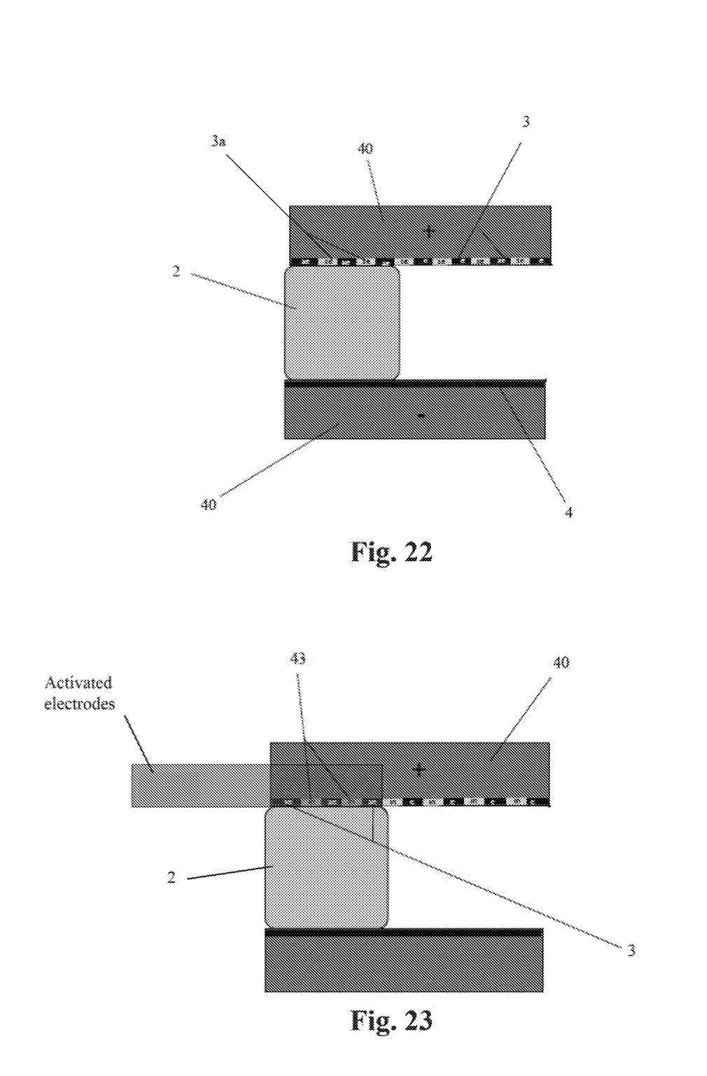

In yet another configuration, with reference to FIG. 22, electrodes may consist of a multiplicity of small electrode members 3 interspersed with sensing electrodes 3a, which can determine, through impedance changes, when they are touching the target tissue 2. Thus, those sensing electrodes 3a not making contact with target tissue 2 indicate as an open circuit, while those sensing electrodes 3a that are making contact with target tissue 2 indicate as a closed circuit. This information may be sent back to SHCU 14, which in turn can direct current to be provided only to those electrodes 3 that are adjacent to sensing electrodes 3a that form a closed circuit. Alternatively, sensing electrodes 3a may be replaced by an insulating material 43, such that the electrodes 3 not touching target tissue 2 will represent an open circuit able to be sensed by the SHCU and not be activated when the pulses are delivered (see FIG. 23).

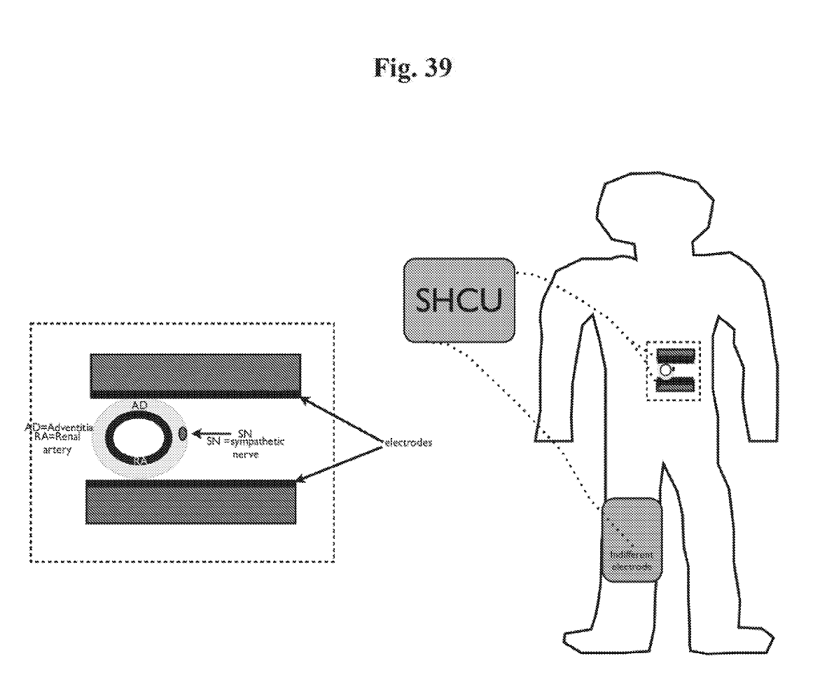

In yet another configuration, shown in FIG. 39, one of electrodes 3, 4 is configured as a unipolar electrode with a remote indifferent electrode as a ground.

Optionally, jaws 40 in any of the configurations described above can be placed through a cannula 44 with a fiber optic scope built into it. Cannula 44 can then be placed through the chest or artery wall to perform the procedure according to the present invention non-invasively (see FIG. 25).

Arrhythmia and Atrial Fibrillation

Any of the embodiments of probe 20 described above may be positioned by the surgeon adjacent the cardiac treatment tissue 2 according to one of several methods. According to one method, the patient is prepared for a MAZE III procedure to the point at which open access to the cardiac region is achieved. When the desired area of the heart is available in the operative field, probes 20 are placed by the surgeon in the planned location to enable the delivery of EMB therapy in accordance with the therapy plan for the treatment.

Alternatively, a minimally invasive surgical approach using a thorascopic procedure may be achieved. This method does not require full open surgical access to the patient's heart; thus, clamp-type probes 20 may be placed on the outer surface of the heart, not in an intravascular location. In this method, the patient is prepared for cardiac surgery in the conventional manner, and general anesthesia is induced. To surgically access the right atrium, the patient is positioned on his or her left side so that the right lateral side of the chest is disposed upward. A wedge or block having a top surface angled at approximately 20-45 degrees can be used and be positioned under the right side of the patient's body so that the right side of his or her body is somewhat higher than the left side. It will be understood, however, that a similar wedge or block can be positioned under the left side of patient when performing the surgical procedure on the left atrium. In either position, the patient's right arm or left arm is allowed to rotate downward to rest on table, exposing either the right lateral side or the left lateral side, respectively of the patient's chest.

In one embodiment of this method, a small incision of about 2-3 cm in length is made between the ribs on the right side of the patient, usually in the third, fourth, or fifth intercostal spaces. When additional maneuvering space is necessary, the intercostal space between the ribs may be widened by spreading of the adjacent ribs. A thoracoscopic access device, including but not limited to a retractor, trocar sleeve, cannula or the like, can provide an access port to the treatment area. The thoracoscopic access device is then positioned in the incision to retract away adjacent tissue and protect it from trauma as instruments are introduced into the chest cavity. Additional thoracoscopic trocars, or the like, can be positioned within intercostal spaces in the right lateral chest inferior and superior to the retractor, as well as in the right anterior (or ventral) portion of the chest if necessary. In other instances, instruments may be introduced directly through small, percutaneous intercostal incisions in the chest.

Once the retractor has been positioned and anchored in the patient's chest, visualization within the thoracic cavity may be accomplished in any of several ways. An endoscope can be positioned through a percutaneous intercostal penetration into the patient's chest, usually through the port of the soft tissue retractor. A video camera can be mounted to the proximal end of the endoscope and is connected to a video monitor for viewing the interior of the thoracic cavity. The endoscope is manipulated to provide a view of the right side of the heart, and particularly, a right side view of the right atrium.

Further, the surgeon may simply view the chest cavity directly through the access port of the retractor. A transesophageal echocardiography can be used, wherein an ultrasonic probe is placed in the patient's esophagus or stomach to ultrasonically image the interior of the heart. A thoracoscopic ultrasonic probe can also be placed through the access device into the chest cavity and adjacent the exterior of the heart for ultrasonically imaging the interior of the heart. An endoscope that has an optically transparent bulb may be used such as an inflatable balloon or transparent plastic lens over the distal end of the scope is introduced into the heart. The balloon can be inflated with a transparent inflation fluid, such as saline, to displace blood away from distal end, and may be positioned against a site such a lesion, allowing the location, shape, and size of an RFEMB lesion to be visualized.

As a further visualization alternative, an endoscope can be utilized which employs a specialized light filter such that only those wavelengths of light not absorbed by blood are transmitted into the heart. The endoscope can have a CCD chip designed to receive and react to such light wavelengths and transmit the image received to a video monitor (i.e., of the SHCU). In this way, the endoscope can be positioned in the heart through the access port and used to see through blood to observe a region of the heart.

The device and system according to the present invention can be used while the heart remains beating. Hence, the trauma and risks associated with cardiopulmonary bypass (CPB) and cardioplegic arrest can be avoided. In other instances, however, arresting the heart may be advantageous. Should it be desirable to place the patient on cardiopulmonary bypass, the patient's right lung is collapsed and the patient's heart is arrested. CPB can be established by introducing a venous cannula into a femoral vein in the patient to withdraw deoxygenated blood therefrom. The venous cannula is connected to a cardiopulmonary bypass system which receives the withdrawn blood, oxygenates the blood, and returns the oxygenated blood to an arterial return cannula positioned in a femoral artery. A pulmonary venting catheter can also be utilized to withdraw blood from the pulmonary trunk. The pulmonary venting catheter can be introduced from the neck through the interior jugular vein and superior vena cava, or from the groin through the femoral vein and inferior vena cava.

For purposes of arresting cardiac function, an aortic occlusion catheter is positioned in a femoral artery by a percutaneous technique such as the Seldinger technique, or through a surgical cut-down. An aortic occlusion catheter is advanced, usually over a guide wire, until an occlusion balloon at its distal end is disposed in the ascending aorta between the coronary ostia and the brachiocephalic artery. Blood can be vented from ascending aorta through a port at the distal end of the aortic occlusion catheter in communication with an inner lumen in the aortic occlusion catheter, through which blood can flow to the proximal end of the catheter. The blood can then be directed to a blood filter/recovery system to remove emboli, and then returned to the patient's arterial system via the CPB system. When it is desired to arrest cardiac function, the occlusion balloon is inflated until it completely occludes the ascending aorta, blocking blood flow there through.

A cardioplegic fluid such as potassium chloride (KCl) can be mixed with oxygenated blood from the CPB system and then delivered to the myocardium in one or both of two ways. Cardioplegic fluid can be delivered in an anterograde manner, retrograde manner, or a combination thereof. In the anterograde delivery, the cardioplegic fluid is delivered from a cardioplegia pump through an inner lumen in the aortic occlusion catheter and the port distal to the occlusion balloon into the ascending aorta upstream of the occlusion balloon. In the retrograde delivery, the cardioplegic fluid can be delivered through a retroperfusion catheter positioned in the coronary sinus from a peripheral vein such as an internal jugular vein in the neck.

With cardiopulmonary bypass established, cardiac function arrested, and the right lung collapsed, the patient is prepared for surgical intervention within the heart. At this point in the procedure, whether cardiac function is arrested and the patient is placed on CPB, or the patient's heart remains beating, the heart treatment procedure and system of the present invention remain substantially similar. The primary difference is that when the procedure of the present invention is performed on an arrested heart, the blood pressure in the internal chambers of the heart is significantly less. It is not necessary to form a hemostatic seal between the device and the heart wall penetration to inhibit blood loss through the penetration thereby reducing or eliminating the need for purse-string sutures around such penetrations.

In order to gain access to the right atrium of the heart, a pericardiotomy is performed using thoracoscopic instruments introduced through the retractor access port. Instruments suitable for use in this procedure, including thoracoscopic angled scissors and thoracoscopic grasping forceps.

After incising a T-shaped opening in the pericardium, about 5.0 cm in length across and about 4.0 cm in length down, the exterior of the heart is sufficiently exposed to allow the closed-chest, closed-heart procedure to be performed. To further aid in visualization and access to the heart, the cut pericardial tissue is retracted away from the pericardial opening with stay sutures extending out of the chest cavity. This technique allows the surgeon to raise and lower the cut pericardial wall in a manner which reshapes the pericardial opening and retracting the heart slightly, if necessary, to provide maximum access for a specific procedure.

Another approach is the trans-vascular approach. There are two procedures of cardiac ablation well known in the art: pulmonary vein ablation for atrial fibrillation and that for other arrhythmias. The invention can be used in accordance with either of these well known procedures.

In treating atrial fibrillation ablation, the procedure well known in the art follows this general format. A balloon catheter (Arctic Front Advance, Medtronic Inc,) with a central lumen is advanced to the opening of the pulmonary vein. Through the central lumen an electro physiologic mapping catheter (Achieve™ Mapping Catheter, Medtronic Inc.) is advanced into the vein. The balloon catheter is inflated in the atrium before being advanced toward the wired vein over the already placed mapping catheter. The balloon is then positioned at the antrum of the pulmonary vein.

Contrast dye is then injected through the guide-wire catheter lumen to assess vein occlusion via fluoroscopy. The therapeutic balloon ablates where the balloon is in contact with the tissue. The anatomical shape and large surface area of the balloon creates circumferential lesions. The mapping catheter is then used to confirm pulmonary vein isolation.

During the catheter ablation procedure, a number of diagnostic catheters (i.e., Stablemapr SM Series Diagnostic Catheters, Medtronics Inc.) are delivered percutaneously through the venous system and placed at key areas of the heart. The catheters have electrodes that are able to sense intra-cardiac electrical signals when connected to the electrophysiology lab system. The resulting electrograms are used to determine the optimal placement of the ablation catheter (5F RF Mariner (Single-Curve) Series Ablation Catheters, Medtronics Inc.). The ablation catheter delivers energy to create a discrete lesion of myocardial scar tissue that eliminates the initiation or propagation of the arrhythmia.

In various embodiments, the system provides the programmatic planning, targeting and delivery of EMB therapy through the placement and use of EMB catheter type probes so as to deliver the planned EMB therapy in a transvascular method as described.

It will be appreciated that the methods and systems of the present invention can be directed to the creation of lesions from the endocardial surfaces of the atria, as well as lesions or portions of the lesions can be created with the endocardial surfaces of the atria.

It will be further appreciated that the methods and systems of the present invention can be utilized to treat atrial fibrillation, Wolfe-Parkinson-White (WPW) Syndrome, ventricular fibrillation, congestive heart failure and other procedures in which interventional devices are introduced into the interior of the heart, coronary arteries, or great vessels. In some embodiments, probes are hand held by the surgeon and do not clamp onto the cardiac tissue but rely on the surgeon for continued therapeutic placement.

Renal Neuromodulation

Known procedures used to prepare a surgical patient for a renal neuromodulation procedure are followed to the point where open access to the renal region is achieved. At that point, in this embodiment, when the desired area of the renal region is available in the operative field, clamping-type probes 20s are placed by the surgeon in the planned location to enable the delivery of EMB therapy in accordance with the therapy plan for the treatment, created by the surgeon using the system in planning mode (described in further detail below).

In various embodiments of the present invention, probe 20, through the use of a pair of electrodes, can take a measurement of the tissue resistance before and after RFEMB treatment. This information can be sent to the SHCU and the adequacy of treatment thusly determined. In another embodiment, the impedance measurements can be used to control the electrical parameters to the tissue to ensure complete EMB in the tissue.

Also in various embodiments, a nerve stimulatory impulse can be delivered by the SHCU to the tissue, looking for a stimulatory sympathetic response such as rise in blood pressure. Such a stimulatory effect could then be tested for again after the procedure to confirm adequate RFEMB ablation.

EMB, by virtue of its bipolar wave forms in the described frequency range, does not cause muscle twitching and contraction. Therefore a procedure using the same may be carried out under local anesthesia without the need for general anesthesia and neuromuscular blockade to attempt to induce paralysis during the procedure. Rather, anesthesia can be applied locally for the control of pain without the need for the deeper and riskier levels of sedation using well known techniques and devices.

One of ordinary skill in the art will understand that the EMB treatment probe(s) 20 may take various forms provided that they are still capable of delivering EMB pulses from the EMB pulse generator 16 of the type, duration, etc. described above.

Software Hardware Control Unit (SHCU) 14 and Treatment System Software

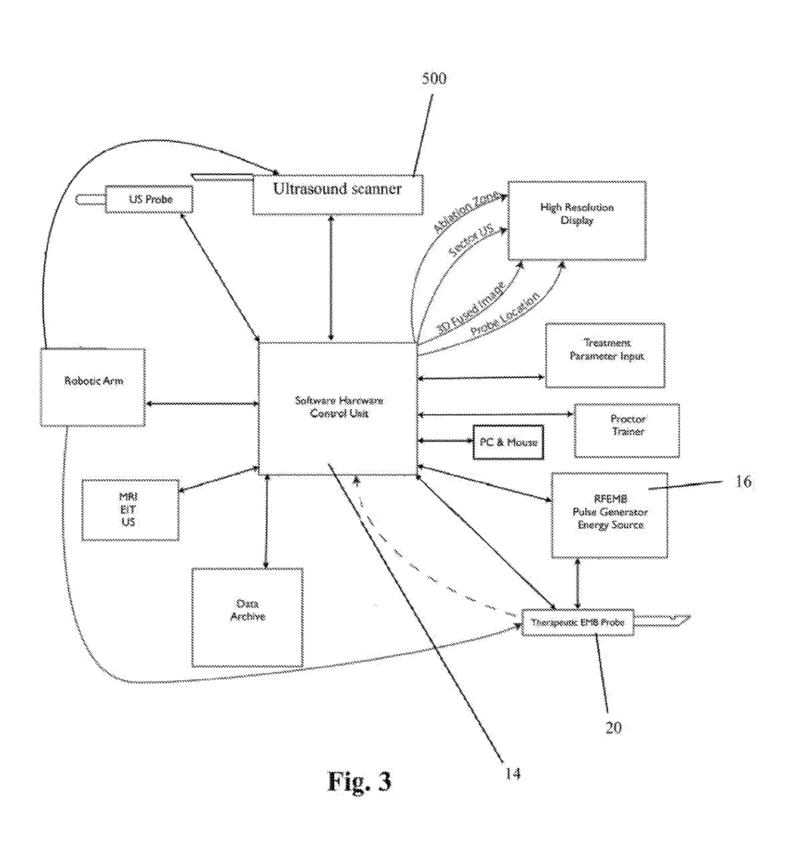

With reference to FIG. 3, the Software Hardware Control Unit (SHCU) 14 is operatively connected to one or more (and preferably all) of the therapeutic and/or diagnostic probes, imaging devices and energy sources described herein: namely, in a preferred embodiment, the SHCU 14 is operatively connected to one or more EMB pulse generator(s) 16, temperature probe(s) 7, and EMB treatment probe(s) 20, via electrical/manual connections for providing power to the connected devices as necessary and via data connections, wired or wireless, for receiving data transmitted by the various sensors attached to each connected device. SHCU 14 is preferably operatively connected to each of the devices described herein such as to enable SHCU 14 to receive all available data regarding the operation and placement of each of these devices.

In an alternative embodiment, SHCU 14 is also connected to one or more of the devices herein via at least one robot arm such that SHCU 14 may itself direct the placement of various aspects of the device relative to a patient, potentially enabling fully automatized and robotic placement and treatment of targeted cardiac or renal nerve tissues via EMB. It is envisioned that the system disclosed herein may be customizable with respect to the level of automation, i.e. the number and scope of components of the herein disclosed method that are performed automatically at the direction of the SHCU 14. At the opposite end of the spectrum from a fully automated system, SHCU 14 may operate software to guide a physician or other operator through a video monitor, audio cues, or some other means, through the steps of the procedure based on the software's determination of the best treatment protocol, such as by directing an operator where to place the EMB treatment probe 20, etc. In each of these variations and embodiments, the system, at the direction of SHCU 14, directs the planning, validation and verification of the Predicted Ablation Zone (to be described in more detail below), to control the application of therapeutic energy to the selected region so as to assure proper treatment, to prevent damage to sensitive structures, and/or to provide tracking, storage, transmission and/or retrieval of data describing the treatment applied.

In a preferred embodiment, SHCU is a data processing system comprising at least one application server and at least one workstation comprising a monitor capable of displaying to the operator a still or video image, and at least one input device through which the operator may provide inputs to the system, i.e. via a keyboard/mouse or touch screen, which runs software programmed to control the system in two “modes” of operation, wherein each mode comprises instructions to direct the system to perform one or more novel features of the present invention. The software according to the present invention may preferably be operated from a personal computer connected to SHCU 14 via a direct, hardwire connection or via a communications network, such that remote operation of the system is possible. The two contemplated modes are Planning Mode and Treatment Mode. However, it will be understood to one of ordinary skill in the art that the software and/or operating system may be designed differently while still achieving the same purposes. In all modes, the software can create, manipulate, and display to the user via a video monitor accurate, real-time three-dimensional images of the human body, which images can be zoomed, enlarged, rotated, animated, marked, segmented and referenced by the operator via the system's data input device(s). As described above, in various embodiments of the present invention the software and SHCU 14 can partially or fully control various attached components, probes, or devices to automate various functions of such components, probes, or devices, or facilitate robotic or remote control thereof.

Planning Mode

The SHCU is preferably operatively connected to one or more external imaging sources such as an magnetic resonance imaging (MRI), ultrasound (US), electrical impedance tomography (EIT), or any other imaging device known in the art and capable of creating images of the human body. Using inputs from these external sources, including specifically imaging of the cardiac or renal vascular area of the patient's bodily structure in the regions requiring treatment, the SHCU first creates one or more “3D Fused Images” of the patient's body in the region of concern. The 3D Fused Images provide a 3D map of the selected treatment area within the patient's body over which locational data obtained from the one or more imaging sources such as an ultrasound scanner according to the present invention may be overlaid to allow the operator to monitor the treatment in real-time against a visual of the actual treatment area.

In a first embodiment, a 3D Fused Image would be created from one or more CT or MRI scans and ultrasound image(s) of the same area of the patient's body. A CT or MRI image used for this purpose may comprise contrast enhanced CT or a multi-parametric magnetic resonance image created using, i.e., any 64 slice scanner commercially available with standard 3D reconstruction software. Alternatively, a standard 3D known in the art can be used for this purpose. An ultrasound image used for this purpose might be the VH® IVUS (intravascular US) Imaging system using the Eagle Eye® Platinum/Platinum ST RX Digital IVUS Catheter.

The ultrasound image may be formed by, i.e., placing an EM field generator (such as that manufactured by Northern Digital Inc.) on the patient, which allows for real-time tracking of a custom ultrasound probe embedded with a passive EM tracking sensor (such as that manufactured by Traxtal, Inc.).

The 3D fused image is then formed by the software according to the present invention by encoding the ultrasound data using position encoded data correlated to the resultant image by its fixed position to the US transducer by the US scanning device. The software according to the present invention also records of the position of any identified areas of concern for later use in guiding therapy.

This protocol thus generates a baseline, diagnostic 3D Fused Image and displays the diagnostic 3D Fused Image to the operator in real time via the SHCU video monitor. Preferably, the system may request and/or receive additional 3D ultrasound images of the treatment area during treatment and fuse those subsequent images with the baseline 3D Fused Image for display to the operator.

As an alternate means of creating the 3D Fused Image, a two-dimensional sweep of the area is performed in the axial plane to render a three-dimensional ultrasound image that is then registered and fused to an MRI or CT of digital fluoroscopy using landmarks common to both the ultrasound image and MRI or CT of digital fluoroscopy image. Areas of concern in the cardiac area and vasculature identified on MRI are semi-automatically superimposed on the real-time US image.

The 3D Fused Image as created by any one of the above methods is then stored in the non-transitive memory of the SHCU, which may employ additional software to locate and electronically tag within the 3D Fused Image specific areas of concern that may require treatment, or its vicinity, including sensitive or critical structures and areas. The SHCU then displays the 3D Fused Image to the operator alone or overlaid with locational data from each of the additional devices described herein where available. The 3D Fused Image may be presented in real time in sector view, or the software may be programmed to provide other views based on design preference.

Upon generation of one or more 3D Fused Images of the planned treatment area and, preferably completion of one or more diagnostic imaging scans of the affected area, the SHCU may display to the operator via a video terminal the precise location(s) of one or more areas of concern which require therapy, via annotations or markers on the 3D Fused Image(s): this area requiring therapy is termed the Target Treatment Zone. This information is then used by the system or by a physician to determine optimal placement of the EMB treatment probe(s) 20. Importantly, the 3D Fused Image should also contain indicia to mark the location of treatment targets designated by the physician which will be used to calculate a path to the treatment area. If necessary due to changes in area or tissue size, the geographic location of each marker can be revised and repositioned, and the 3D Fused Image updated in real time by the software, using 3D ultrasound data as described above. The system may employ an algorithm for detecting changes in target tissue size and requesting additional ultrasound scans, and may request ultrasound scans on a regular basis, or the like.

In a preferred embodiment, the software may provide one or more “virtual” EMB treatment catheter type probes 20 which may be overlaid onto the 3D Fused Image showing the areas of concern by the software or by the treatment provider to determine the extent of ablation that would be accomplished with each configuration. Preferably, the software is configured to test several possible probe 20 placements and calculate the probable results of treatment to the affected area via such a probe 20 (the Predicted Ablation Zone) placement using a database of known outcomes from various EMB treatment protocols or by utilizing an algorithm which receives as inputs various treatment parameters such as pulse number, amplitude, pulse width and frequency. By comparing the outcomes of these possible probe locations to the targeted tissue volume as indicated by the 3D Fused Image, the system may determine the optimal probe 20 placement. Alternatively, the system may be configured to receive inputs from a physician to allow him or her to manually arrange and adjust the virtual EMB treatment probes to adequately cover the treatment area and volume based on his or her expertise.

When the physician is satisfied with the Predicted Ablation Zone coverage shown on the Target Treatment Zone based on the placement and configuration of the virtual EMB treatment probes as determined by the system or by the physician himself, the physician “confirms” in the system (i.e. “locks in”) the three-dimensional placement and energy/medication delivery configuration of the virtual EMB treatment probes and the system registers the position of each as an actual software target to be overlaid on the 3D Fused Image and used by the system for guiding the placement of the real probe(s) according to the present invention (which may be done automatically by the system via robotic arms or by the physician by tracking his or her progress on the 3D Fused Image).

If necessary, EMB treatment, as described in further detail below, may be carried out immediately after the planning of therapy is completed for the patient. Alternately, the EMB treatment plan can be created in one session and stored for later use so that EMB therapy may take place days or even weeks later. In the latter case, the steps described with respect to the Planning Mode, above, may be undertaken by the software/physician at any point.

Treatment Mode

The software displays, via the SHCU video monitor, the previously confirmed and “locked in” Target Treatment Zone, Predicted Ablation Zone and 3D Fused Image, with the location and configuration of all previously confirmed virtual probes and their calculated configuration and placement in the targeted locations, which can be updated as needed at time of treatment to reflect any required changes as described above.

The system displays the Predicted Ablation Zone and the boundaries thereof as an overlay on the 3D Fused Image including the Target Treatment Zone and directs the physician (or robotic arm) as to the targeted placement of each EMB treatment probe 20. The Predicted Ablation Zone may be updated and displayed in real time as the physician positions each probe 20 to give graphic verification of the boundaries of the Target Treatment Zone, allowing the physician to adjust and readjust the positioning of the Therapeutic EMB Probes, sheaths, electrode exposure and other treatment parameters (which in turn are used to update the Predicted Ablation Zone). When the physician (or, in the case of a fully automated system, the software) is confident of accurate placement of the probes, he or she may provide such an input to the system, which then directs the administration of EMB pulses via the EMB pulse generator 16 and probes 20.

The SHCU controls the pulse amplitude 30, frequency 31, polarity and shape provided by the EMB pulse generator 16, as well as the number of pulses 32 to be applied in the treatment series or pulse train, the duration of each pulse 32, and the inter pulse burst delay 33. Although only two are depicted in FIG. 10 due to space constraints, EMB ablation is preferably performed by application of a series of not less than 100 electric pulses 32 in a pulse train so as to impart the energy necessary on the target tissue 2 without developing thermal issues in any clinically significant way. The width of each individual pulse 32 is preferably from 100 to 1000 μs with an inter pulse burst interval 33 during which no voltage is applied in order to facilitate heat dissipation and avoid thermal effects. The relationship between the duration of each pulse 32 and the frequency 31 (period) determines the number of instantaneous charge reversals experienced by the cell membrane during each pulse 32. The duration of each inter pulse burst interval 33 is determined by the controller 14 based on thermal considerations. In an alternate embodiment the system is further provided with a temperature probe 22 inserted proximal to the target tissue 2 to provide a localized temperature reading at the treatment site to the SHCU 14. The temperature probe 22 may be a separate, needle type probe having a thermocouple tip, or may be integrally formed with or deployed from one or more of the electrodes or Therapeutic EMB Probes. The system may further employ an algorithm to determine proper placement of this probe for accurate readings from same. With temperature feedback in real time, the system can modulate treatment parameters to eliminate thermal effects as desired by comparing the observed temperature with various temperature set points stored in memory. This is very important to prevent thermal injury to the heart or nerve vessel wall. More specifically, the system can shorten or increase the duration of each pulse 32 to maintain a set temperature at the treatment site to, for example, create a heating (high temp) for the probe tract to prevent bleeding or to limit heating (low temp) to prevent any coagulative necrosis. The duration of the inter pulse burst interval can be modulated in the same manner in order to eliminate the need to stop treatment and maximizing the deposition of energy to accomplish EMB. Pulse amplitude 30 and total number of pulses in the pulse train may also be modulated for the same purpose and result.

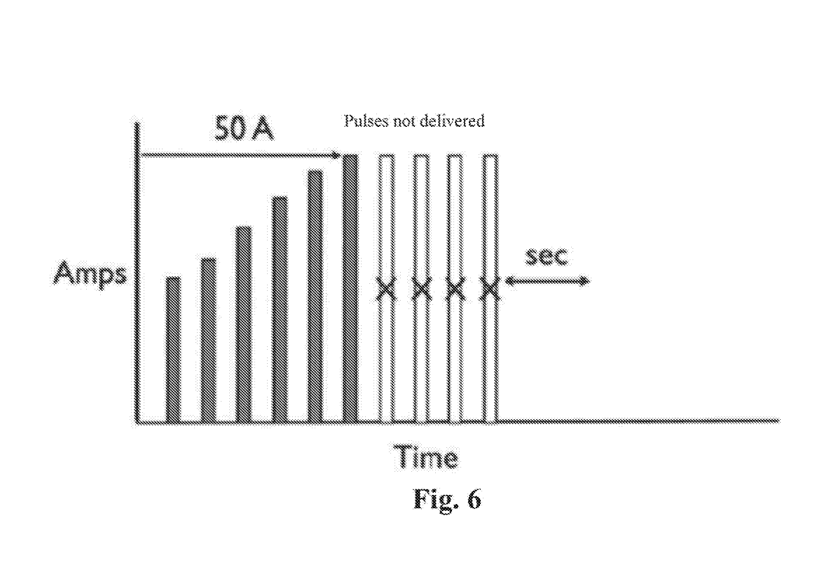

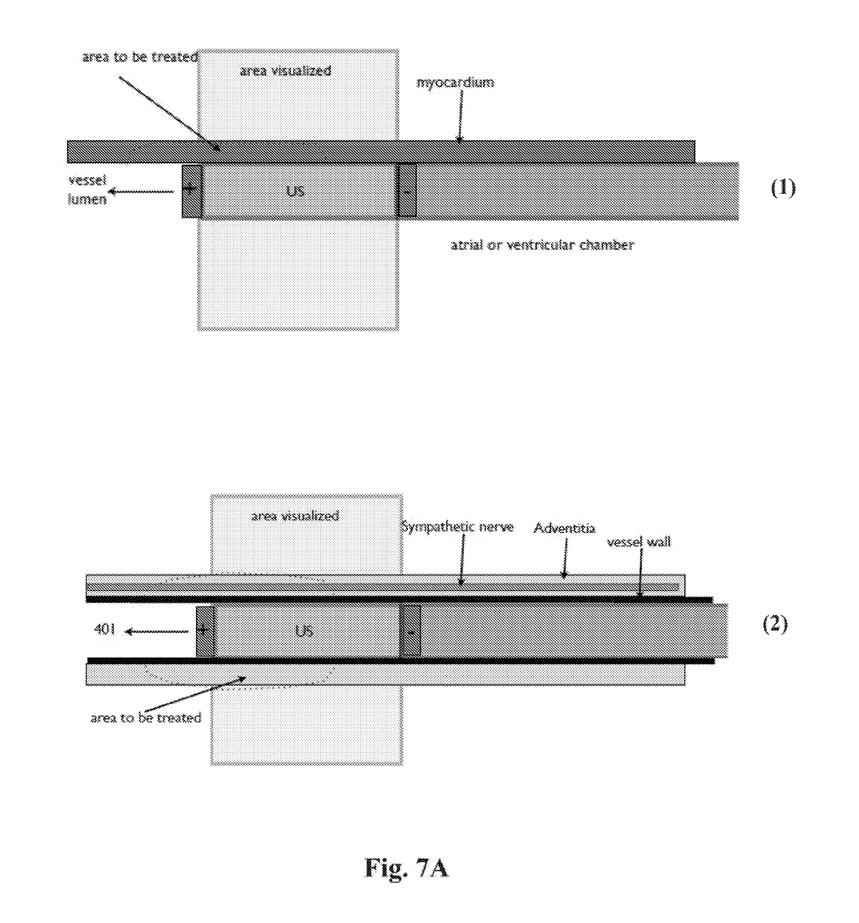

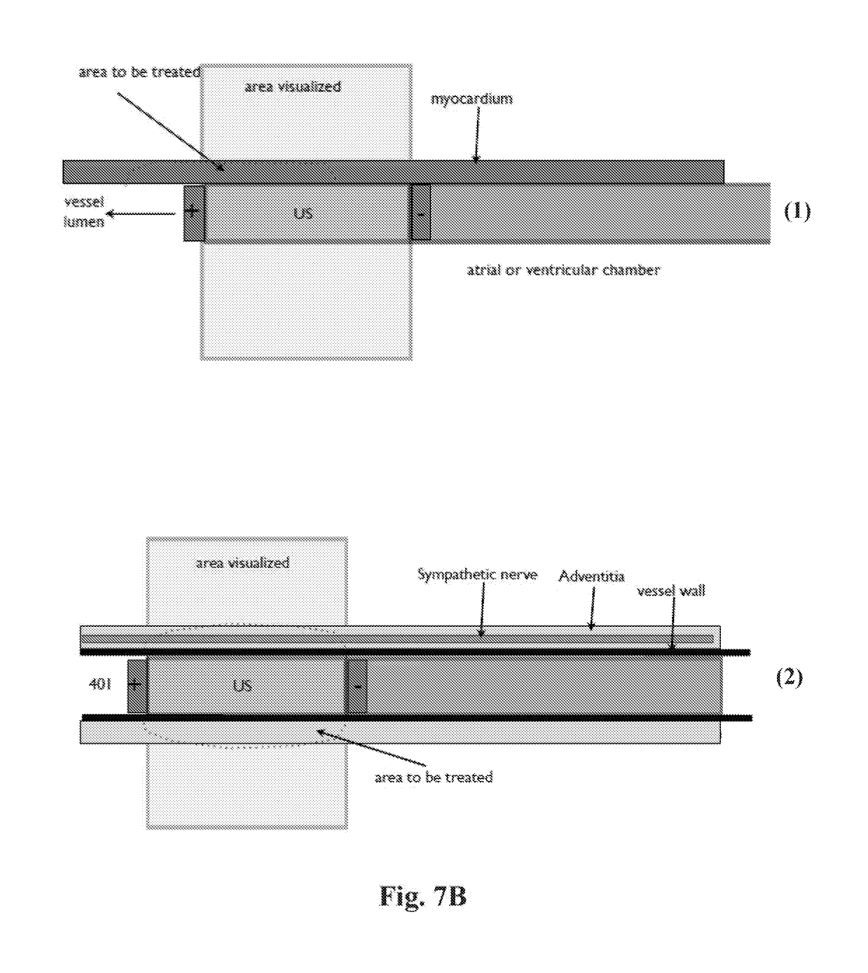

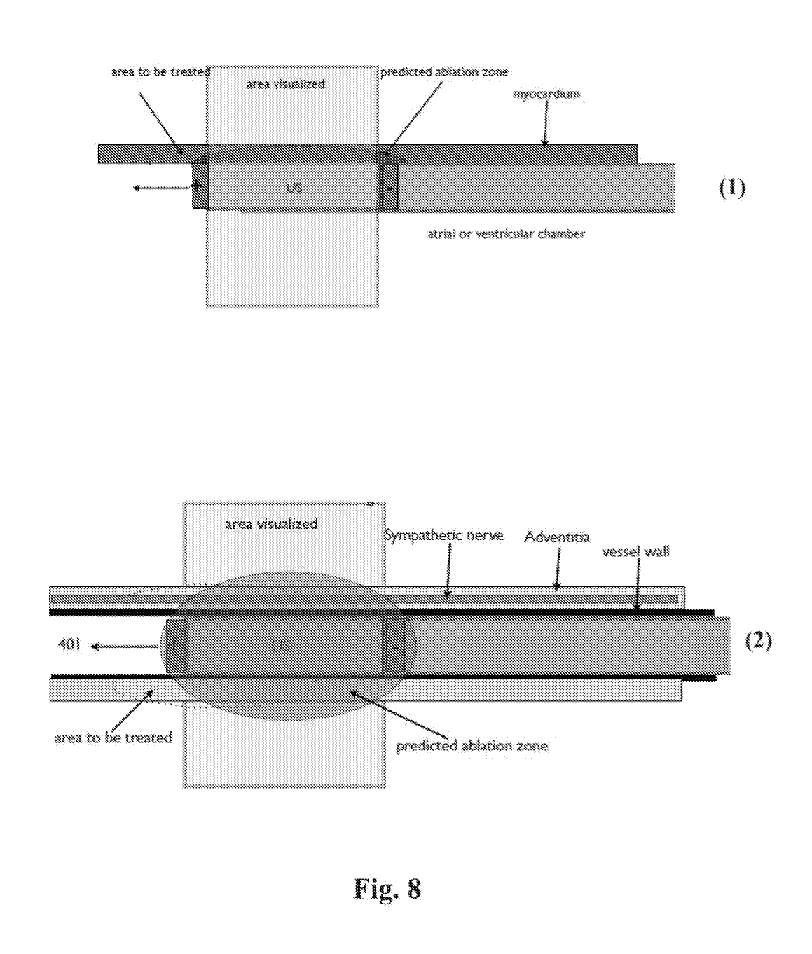

In yet another embodiment, the SHCU may monitor or determine current flow through the tissue during treatment for the purpose of avoiding overheating while yet permitting treatment to continue by reducing the applied voltage. Reduction in tissue impedance during treatment due to charge buildup and membrane rupture can cause increased current flow which engenders additional heating at the treatment site. With reference to FIG. 6, prior treatment methods have suffered from a need to cease treatment when the current exceeds a maximum allowable such that treatment goals are not met. As with direct temperature monitoring, the present invention can avoid the need to stop treatment by reducing the applied voltage and thus current through the tissue to control and prevent undesirable clinically significant thermal effects. Modulation of pulse duration and pulse burst interval duration may also be employed by the controller 14 for this purpose as described.

During treatment, the software captures all of the treatment parameters, all of the tracking data and representational data in the Predicted Ablation Zone, the Target Treatment Zone and in the 3D Fused Image as updated in real time to the moment of therapeutic trigger. Based on the data received by the system during treatment, the treatment protocol may be adjusted or repeated as necessary.

The software may also store, transmit and/or forwarding treatment data to a central database located on premises in the physician's office and/or externally via a communications network so as to facilitate the permanent archiving and retrieval of all procedure related data. This will facilitate the use and review of treatment data, including for diagnostic purposes and pathology related issues, for treatment review purposes and other proper legal purposes including regulatory review.

The software may also transmit treatment data in real time to a remote proctor/trainer who can interact in real time with the treating physician and all of the images displayed on the screen, so as to insure a safe learning experience for an inexperienced treating physician, and so as to archive data useful to the training process and so as to provide system generated guidance for the treating physician. In another embodiment, the remote proctor can control robotically all functions of the system.

Optionally, with one or more EMB treatment probes 20 still in place within the ablated tissue, the physician or system can perform injection of medicines, agents, or other materials into the ablated tissue, using capabilities built into the probe, as described above, or through separate delivery means.

In other embodiments of the present invention, some or all of the treatment protocol may be completed by robotic arms, which may include an ablation probe guide which places the specially designed Therapeutic EMB Probe in the correct targeted location relative to the targeted tissue. Robotic arms may also be used to hold the US transducer in place and rotate it to capture images for a 3D US reconstruction.

In addition, the robotic arm can hold the Therapeutic EMB Probe itself and can directly insert the probe into the targeted location selected for treatment using and reacting robotically to real time positioning data supported by the 3D Fused Image and Predicted Ablation Zone data and thereby achieving full placement robotically.

Robotic components capable of being used for these purposes include the iSR′obot™ Mona Lisa robot, manufactured by Biobot Surgical Pte. Ltd. In such embodiments the Software supports industry standard robotic control and programming languages such as RAIL, AML, VAL, AL, RPL, PYRO, Robotic Toolbox for MATLAB and OPRoS as well as other robot manufacturer's proprietary languages.

The SHCU can fully support Interactive Automated Robotic Control through a proprietary process for image sub-segmentation of the targeted tissue and nearby sensitive anatomical structures for planning and performing robotically guided therapeutic intervention.

Sub-segmentation is the process of capturing and storing precise image detail of the location size and placement geometry of the described anatomical object so as to be able to define, track, manipulate and display the object and particularly its three-dimensional boundaries and accurate location in the body relative to the rest of the objects in the field and to the anatomical registration of the patient in the system so as to enable accurate three-dimensional targeting of the object or any part thereof, as well as the three-dimensional location of its boundaries in relation to the locations of all other subsegmented objects and computed software targets and probe pathways. The software sub-segments out various critical substructures, in the treatment region, in a systematic and programmatically supported and required fashion, which is purposefully designed to provide and enable the component capabilities of the software as described herein.

Having now fully set forth the preferred embodiment and certain modifications of the concept underlying the present invention, various other embodiments as well as certain variations and modifications of the embodiments herein shown and described will obviously occur to those skilled in the art upon becoming familiar with said underlying concept. It is to be understood, therefore, that the invention may be practiced otherwise than as specifically set forth herein.

### STATEMENT OF INDUSTRIAL APPLICABILITY

Atrial fibrillation and the reduction of sympathetic renal nerve activity are different but related conditions that may be treated with ablation of cardiac or sympathetic renal nerve tissue, respectively. However, current treatments for both of these conditions involve major risks such as the invasive nature of the treatment or the requirement for a patient to be placed under general anesthesia to receive treatment. There would be great industrial applicability in an effective ablation technique adaptable for treatment of atrial fibrillation and achieving renal neuromodulation that was minimally invasive and less traumatic than classic methods of ablation, and which could be conducted without the need for general anesthesia, which may have dangerous side effects. The instant invention fulfills this need by utilizing Radio-Frequency Electrical Membrane Breakdown to destroy the cellular membranes of unwanted tissue without denaturing the intra-cellular contents of the cells comprising the tissue.

We claim:

1. A method of ablating soft tissue in a living subject using radio frequency electrical membrane breakdown, the method comprising:
identifying a location of said soft tissue within said subject;
introducing at least one electrode to said location within said subject; and
applying to said soft tissue at said location, via said at least one electrode, an electric field sufficient to cause electrical membrane breakdown of a cell membrane of a plurality of cells of said soft tissue to cause immediate spillage of all intracellular components into an extracellular space and exposure of an internal constituent part of said cell membrane to said extracellular space;
wherein said method is performed in a minimally-invasive manner.

2. The method of claim 1, wherein said soft tissue comprises tissue of the renal sympathetic nerve.

3. The method of claim 2, further comprising the step of taking one or more impedance measurements at said location.

4. The method of claim 3, wherein said step of taking one or more impedance measurements at said location occurs simultaneously with said step of applying said electric field.

5. The method of claim 3, wherein said step of taking one or more impedance measurements at said location occurs both before and after said step of applying said electric field.

6. The method of claim 2, wherein said method is carried out bilaterally on both a left and a right kidney of said living subject.

7. The method of claim 2, wherein said step of applying said electric field further comprises configuring said electric field to be aligned with a longer dimension of one or more cells in said soft tissue.

8. The method of claim 2, wherein said method is applied from a percutaneous catheter approach.

9. The method of claim 2, further comprising delivering a stent in a renal blood vessel of said living subject proximate said location.

10. The method of claim 1, wherein said method is conducted under ultrasound guidance.

11. The method of claim 2, wherein said method is performed without the use of general anesthesia.

12. The method of claim 1, wherein said soft tissue comprises cardiac tissue.

13. The method of claim 12, wherein said method is conducted using a thorascopic procedure.

14. The method of claim 12, wherein said method is performed without arresting a heartbeat of said living subject.

15. The method of claim 12, wherein said method is conducted using a transvascular approach.

16. A system for ablating soft tissue in a living subject using radio frequency electrical membrane breakdown, the system comprising:
at least one EMB pulse generator capable of generating an electric field sufficient to cause electrical membrane breakdown of a cell membrane of a plurality of cells of said soft tissue to cause immediate spillage of all intracellular components into an extracellular space and exposure of an internal constituent part of said cell membrane to said extracellular space;
at least one EMB treatment probe capable of delivering said electric field to said soft tissue; and
at least one software hardware control unit operatively connected to said at least one EMB pulse generator and said at least one EMB treatment probe;
wherein said soft tissue is selected from the group comprising tissue of the renal sympathetic nerve or cardiac tissue.

17. The system of claim 16, wherein said at least one EMB treatment probe is an angiographic balloon dilatation catheter.

18. The system of claim 16, wherein said at least one EMB treatment probe comprises at least two electrodes mounted on an outer surface of said at least one EMB treatment probe, at least one of said at least two electrodes being a positive electrode and at least one of said two electrodes being a negative electrode, and wherein said at least two electrodes are spaced apart from one another by a predetermined space.

19. The system of claim 18, wherein an ultrasound transducer is located on said at least one EMB treatment probe in said predetermined space between said at least two electrodes.

20. The system of claim 17, wherein said at least one EMB treatment probe is a clamp-type probe comprising two jaws, and wherein a first one of said jaws comprises a positive electrode and a second one of said jaws comprises a negative electrode.

21. The system of claim 17, wherein said at least one EMB treatment probe comprises a balloon at a distal end thereof.

22. The system of claim 21, wherein said balloon comprises at least one positive electrode and at least one negative electrode on an outer surface thereof.

23. The system of claim 17, wherein said at least one EMB treatment probe comprises a plurality of positive electrodes and a plurality of negative electrodes, each one of said plurality of positive electrodes being spaced apart from a next nearest one of said plurality of positive electrodes by one of said plurality of negative electrodes.

24. The system of claim 17, wherein said at least one EMB treatment probe is configured as a unipolar electrode with a remote indifferent electrode as a ground.
